(19) United States
(12) Patent Application Publication
(10) Pub. No.: US 2024/0400680 A1
(43) Pub. Date: Dec. 5, 2024

(54) FUSION PROTEIN OF ANTI-TIGIT ANTIBODY AND IL2 OR VARIANT THEREOF AND APPLICATION THEREOF

(71) Applicant: SHANGHAI CELGEN BIO-PHARMACEUTICAL CO., LTD., SHANGHAI (CN)

(72) Inventors: Yi CHEN, SHANGHAI (CN); Zeling CAI, SHANGHAI (CN); Qiaoqiao HE, SHANGHAI (CN); Yaling WANG, SHANGHAI (CN)

(21) Appl. No.: 18/694,865

(22) PCT Filed: May 17, 2022
(86) PCT No.: PCT/CN2022/093346

§ 371 Date: Mar. 22, 2024

(30) Foreign Application Priority Data
Sep. 22, 2021 (CN) 202111105544.0

(51) Int. Cl. C07K 16/28; A61P 35/00; C07K 14/55

(52) U.S. Cl. CPC C07K 16/2803; A61P 35/00; C07K 14/55; C07K 2317/52; C07K 2317/565; C07K 2317/732; C07K 2317/734; C07K 2317/76; C07K 2317/92; C07K 2319/00

(57) ABSTRACT

Provided are a fusion protein of an anti-TIGIT antibody and IL2 or a variant thereof, and an application thereof. Specifically, a fusion protein is provided, which comprises: (a) a first polypeptide comprising an anti-TIGIT antibody or an antigen-binding fragment thereof; and (b) a second polypeptide comprising interleukin-2 (IL-2) or a variant thereof having a lymphocyte growth promoting activity, the second polypeptide being fused to the first polypeptide. Also provided are an application of such a fusion protein in positively regulating immune cell activity and/or improving immune responses; and/or, an application thereof in the treatment of cancer, immunodeficiency or inflammatory diseases, or infectious diseases.

### TECHNICAL FIELD

The application relates to the fields of biotechnology and medicine. Specifically, the application relates to antibody-interleukin fusion proteins, in particular, those comprising an antibody capable of specifically binding to TIGIT and interleukin-2 (IL-2) or various variants thereof. Furthermore, the application involves polynucleotides encoding such fusion proteins, vectors and host cells for expressing these fusion proteins. The application also relates to methods for producing and preparing such fusion proteins, as well as pharmaceutical compositions and therapeutic methods for treating diseases.

### BACKGROUND

Interleukin-2 (IL2) is a T cell growth factor induced by antigen stimulation, which can activate and promote the proliferation and differentiation of T cells, maintaining the growth and proliferation of several immune cells such as B cells, natural killer cells, and macrophages. Therefore, IL2 plays a significant role in the treatment of tumors and immunodeficiency diseases, and is one of the first FDA-approved immunotherapeutic drugs for cancer.

In recent years, though wild-type IL2 has achieved remarkable results in cancer treatment, its short half-life in vivo, along with potential side effects such as fever, vomiting, diarrhea, dizziness, and hypotension, have shifted research focus towards new mutant IL2 proteins with stronger specificity and better therapeutic effects.

IL2 exerts its biological activity by binding to the IL2 receptor (IL2R) on the cell membrane. IL2R is a complex consisting of α (55 kd), β (75 kd), and γ (64 kd) chains. Only when all three chains are present, IL2 can bind to IL2R with high affinity (Kd≈10⁻¹¹M). Cells expressing merely the β and γ chains but lacking the α chain can be capable of signaling but can only bind to IL2 with moderate affinity (Kd≈10⁻⁹M). Cells expressing only the α chain bind to IL2 with low affinity (Kd≈10⁻⁸M), and fail in signaling. The γ subunit alone does not bind to IL2. High-affinity IL2Rs are mainly found on activated T cells, B lymphocytes, and NK cells, while most resting NK cells and macrophages express moderate-affinity IL2Rs, and low-affinity IL2Rs are expressed on resting T cells (GAFFEN S L et al., Cytokine, 2004, 28 (3): 109-123).

Studies have found that mutations in IL2 protein can enhance its affinity for IL2RB. This new mutant IL2 protein, compared to wild-type IL2, can better induce the proliferation of cytotoxic T cells and reduce the expansion of Treg cells, thereby improving anti-tumor effects (Levin et al., Nature 484: 529 (2012)).

T cell immunoreceptor with immunoglobulin and ITIM domains (TIGIT, also known as WUCAM, Vstm3, or VSIG9) is a checkpoint molecule primarily expressed on the surface of immune cells such as NK cells, T cells, and Treg cells. It comprises an immunoreceptor tyrosine-based inhibitory motif (ITIM) in its cytoplasmic tail and is a typical inhibitory receptor protein (Yu et al., Nat. Immunol. 10:48-57, 2009).

TIGIT can inhibit the body's immune response through various mechanisms. By targeting molecules in the T cell antigen receptor (TCR) signaling pathway, TIGIT directly blocks the activation, proliferation, and acquisition of effector functions of initial T cells (Tn), and can also inhibit the prolifeation of CD4+ T cells and the production of inflammatory cytokines (Fourcade J et al., JCI Insight, 2018, 3 (14): e121157). TIGIT can indirectly inhibit T cells by regulating the cytokine production of dendritic cells (Yu et al., Nat. Immunol. 10:48-57, 2009). TIGIT can also enhance the stability of Tregs and their inhibitory function on the proliferation of IFN-γ-producing T cells (Fourcade J et al., JCI Insight, 2018, 3 (14): e121157).

In the tumor environment, TIGIT is highly expressed on the surface of NK cells and effector T cells associated with tumor infiltration or migration, while its ligand CD155 is highly expressed on tumor cell surfaces. Tumor cells directly act on NK cells and effector T cells through the binding of CD155/TIGIT, inhibiting their activities (Li et al., J. Biol. Chem. 289:17647-17657, 2014).

TIGIT is a promising new immunotherapy target for enhancing immune responses and for the prevention and/or treatment of tumors, infections, or infectious diseases. CD155-blocking antibodies that target TIGIT, especially anti-human TIGIT antibodies, can release the tumor killing activity of immune effector cells, and is promising for achieving good anti-tumor efficacy.

### SUMMARY OF THE INVENTION

The application provides a TIGIT-targeting CD155-blocking antibody and its corresponding fusion proteins.

In a first aspect, provided is an anti-TIGIT antibody or antigen-binding fragment thereof.

In some embodiments, the anti-TIGIT antibody is a monoclonal antibody.

In some embodiments, the anti-TIGIT antibody or antigen-binding fragment thereof is selected from the group consisting of:
(a) The anti-TIGIT antibody or antigen-binding fragment thereof that competitively binds to TIGIT with CD155 and has a binding affinity to human TIGIT with an EC50 of 0.01 to 20 nM; and/or
(b) The anti-TIGIT antibody or antigen-binding fragment thereof including heavy chain complementarity-determining regions (VH CDRs) 1 to 3 and light chain complementarity-determining regions (VL CDRs) 1 to 3 selected from the group consisting of VH CDR1: SEQ ID NO: 81 or 89; VH CDR2: SEQ ID NO: 82 or 90; VH CDR3: SEQ ID NO: 83 or 91; VL CDR1: SEQ ID NO: 85 or 93; VL CDR2: SEQ ID NO: 86 or 94; and VL CDR3: SEQ ID NO: 87 or 95; and/or
(c) The anti-TIGIT antibody or antigen-binding fragment thereof including the anti-TIGIT antibody or antigen-binding fragment thereof with amino acid sequences of VH CDRs 1 to 3 as follows: SEQ ID NOs: 81, 82, and 83; or SEQ ID NOs: 89, 90, and 91; and the amino acid sequences of VL CDRs 1 to 3 as follows: SEQ ID NOs: 85, 86, and 87; or SEQ ID NOs: 93, 94, and 95; and/or
(d) The anti-TIGIT antibody or antigen-binding fragment thereof including VH CDRs 1 to 3 and VL CDRs 1 to 3 selected from the group consisting of SEQ ID NOs: 81, 82, and 83 and SEQ ID NOs: 85, 86, and 87; or SEQ ID NOs: 89, 90, and 91 and SEQ ID NOs: 93, 94, and 95; and/or
(e) The anti-TIGIT antibody or antigen-binding fragment thereof including a heavy chain variable region VH selected from the group consisting of SEQ ID NO: 80 or 88; and/or a light chain variable region VL selected from the group consisting of SEQ ID NO: 84 or 92; and/or
(f) The anti-TIGIT antibody or antigen-binding fragment thereof including a combination of VH and VL selected from the group consisting of VH of SEQ ID NO: 80 and VL of SEQ ID NO: 84; or VH of SEQ ID NO: 88 and VL of SEQ ID NO: 92, or
a sequence having at least 95% sequence identity thereto.

In some embodiments, the anti-TIGIT antibody or antigen-binding fragment thereof comprises the IgG1 gamma constant region (e.g., the IgG1 gamma constant region with the UniProtKB accession number P01857). In some embodiments, the constant region has characteristics selected from the group consisting of: (a) the constant region comprises no mutations; (b) the constant region comprises one or more mutations that reduce the antibody-mediated ADCC and CDC activities, such as the aminod acid mutations D265A and N297G; (c) the constant region comprises paired heavy chain constant regions (CHs), one of which comprises mutations that introduce a knob structure (e.g., mutations S354C and/or T366W) and the other comprises mutations that introduce a hole structure (e.g., Y349C, T366S, L368A, and/or Y407V), and wherein the knob structure matches the hole structure to form a stable dimer; (d) the constant region comprises paired CHs, one of which comprises amino acid mutations that introduce a positive charge (e.g., mutations E356K and H435R) and the other comprises amino acid mutations that introduce a negative charge (e.g., K439E), and wherein a stable dimer is formed through charge interaction. The positions of the above mutations are based on the EU numbering.

In some aspects, provided is a fusion protein, comprising: (a) a first polypeptide, which comprises an anti-TIGIT antibody or antigen-binding fragment thereof; (b) a second polypeptide, which comprises interleukin-2 (IL-2) or a variant thereof having lymphocyte growth promoting activity, wherein the second polypeptide is fused to the first polypeptide.

In some embodiments, the first polypeptide comprises an anti-TIGIT antibody or antigen-binding fragment thereof selected from the group consisting of:
(a) the anti-TIGIT antibody or antigen-binding fragment thereof that competitively binds to TIGIT with CD155 and has a binding affinity to human TIGIT with an EC50 of 0.01 to 20 nM; and/or
(b) the anti-TIGIT antibody or antigen-binding fragment thereof comprising heavy chain complementarity-determining regions (VH CDRs) 1 to 3 and light chain complementarity-determining regions (VL CDRs) 1 to 3 selected from the group consisting of: VH CDR1 selected from SEQ ID NO: 81 or 89; VH CDR2 selected from SEQ ID NO: 82 or 90; VH CDR3 selected from SEQ ID NO: 83 or 91; VL CDR1 selected from SEQ ID NO: 85 or 93; VL CDR2 selected from SEQ ID NO: 86 or 94; and VL CDR3 selected from SEQ ID NO: 87 or 95; and/or
(c) the anti-TIGIT antibody or antigen-binding fragment thereof comprising amino acid sequences for VH CDRs 1 to 3 as follows: SEQ ID NOs: 81, 82, and 83; or SEQ ID NOs: 89, 90, and 91; and amino acid sequences for VL CDRs 1 to 3 as follows: SEQ ID NOs: 85, 86, and 87; or SEQ ID NOs: 93, 94, and 95; and/or
(d) the anti-TIGIT antibody or antigen-binding fragment thereof comprising VH CDRs 1 to 3 and VL CDRs 1 to 3 selected from the group consisting of: SEQ ID NOs: 81, 82, and 83 and SEQ ID NOs: 85, 86, and 87; or SEQ ID NOs: 89, 90, and 91 and SEQ ID NOs: 93, 94, and 95; and/or
(e) the anti-TIGIT antibody or antigen-binding fragment thereof comprising a heavy chain variable region VH selected from SEQ ID NO: 80 or 88; and/or a light chain variable region VL selected from SEQ ID NO: 84 or 92; and/or
(f) the anti-TIGIT antibody or antigen-binding fragment thereof comprising a combination of VH and VL selected from: VH of SEQ ID NO: 80 and VL of SEQ ID NO: 84; or VH of SEQ ID NO: 88 and VL of SEQ ID NO: 92, or
a sequence having at least 95% sequence identity thereto.

In some embodiments, the first polypeptide comprises the IgG1 gamma constant region (e.g., the IgG1 gamma constant region with the UniProtKB accession number P01857), wherein the constant region has characteristics selected from the group consisting of:
(a) The constant region comprises no mutations;
(b) The constant region comprises one or more mutations that reduce the antibody-mediated ADCC and CDC activities, such as the amino acid mutations D265A and N297G;
(c) The constant region comprises a pair of heavy chain constant regions (CHs), one of which includes a mutation that introduces a knob structure (e.g., mutations S354C and/or T366W), and the other comprises a mutation that introduces a hole structure (e.g., Y349C, T366S, L368A, and/or Y407V), wherein the knob structure matches the hole structure to form a stable dimer;
(d) The constant region comprises a pair of CHs, one of which includes an amino acid mutation that introduces a positive charge (e.g., mutations E356K and H435R), and the other comprises an amino acid mutation that introduces a negative charge (e.g., K439E), wherein a stable dimer is formed through charge interaction,
with the positions of the above mutations being based on the EU numbering.

In some embodiments, the second polypeptide has one or more characteristics selected from the group consisting of:
(a) The second polypeptide further comprises a signal peptide;
(b) The IL-2 is wild-type IL-2 or a functional fragment thereof, for example, derived from humans, primates, or rodents;
(c) Compared to wild-type IL-2, the IL-2 variant has an increased binding affinity to the IL-2Rβ subunit and/or a decreased binding affinity to the IL-2Rα subunit;
(d) Compared to wild-type IL-2, the IL-2 variant has unchanged or enhanced lymphocyte growth promoting activity.

In some embodiments, the IL-2 variant comprises one or more mutations selected from the group consisting of: L80F, R81D, L85V, 186V, 192F, F42A, for example, comprising the mutation combination of L80F, R81D, L85V, 186V, and 192F and/or mutation F42A, wherein the positions of the above mutations are based on the EU numbering.

In some embodiments, the first and second polypeptides are connected via a linker, for example, the linker is a glycine linker, such as Gₙ, or a glycine/serine linker, such as the amino acid sequences (GS)ₙ, (GGS)ₙ, (GGGS)ₙ, (GGGGS)ₙ, or (GGGGGS)ₙ, wherein n is an integer of 1, 2, 3, 4, 5, 6, 7, 8, 9, or 10.

In some embodiments, the second polypeptide is connected to the N-terminus and/or C-terminus of the antibody heavy chain in the first polypeptide.

In some embodiments, the heavy chain of the first polypeptide is fused to the second polypeptide to form an amino acid sequence selected from the group consisting of: SEQ ID NOs: 39, 51, 44, 55, 46, 57, 66, 72, 68, 74, 70, 76, 42, 53, 45, 56, 49, 59, 67, 73, 69, 75, 71, and 77, or a sequence having at least 80% sequence identity thereto.

In some embodiments, if present, the heavy chain in the fusion protein not fused to the second polypeptide has an amino acid sequence selected from the group consisting of: SEQ ID NOs: 40, 52, 47, 58, 43, 54, 50, and 60, or a sequence having at least 80% sequence identity thereto.

In some embodiments, if present, the light chain in the fusion protein has an amino acid sequence selected from the group consisting of: SEQ ID NOs: 41 and 48, or a sequence having at least 80% sequence identity thereto.

In some embodiments, the fusion protein comprises a sequence combination selected from the group consisting of: (a) SEQ ID NOs: 39, 40, and 41; (b) SEQ ID NOs: 51, 52, and 41; (c) SEQ ID NOs: 44, 40, and 41; (d) SEQ ID NOs: 55, 52, and 41; (e) SEQ ID NOs: 46, 47, and 48; (f) SEQ ID NOs: 57, 58, and 48; (g) SEQ ID NOs: 66, 40, and 41; (h) SEQ ID NOs: 72, 52, and 41; (i) SEQ ID NOs: 68, 40, and 41; (j) SEQ ID NOs: 74, 52, and 41; (k) SEQ ID NOs: 70, 47, and 48; (1) SEQ ID NOs: 76, 58, and 48; (a′) SEQ ID NOs: 42, 43, and 41; (b′) SEQ ID NOs: 53, 54, and 41; (c′) SEQ ID NOs: 45, 43, and 41; (d′) SEQ ID NOs: 56, 54, and 41; (e′) SEQ ID NOs: 49, 50, and 48; (f) SEQ ID NOs: 59, 60, and 48; (g′) SEQ ID NOs: 67, 43, and 41; (h′) SEQ ID NOs: 73, 54, and 41; (i′) SEQ ID NOs: 69, 43, and 41; (j′) SEQ ID NOs: 75, 54, and 41; (k′) SEQ ID NOs: 71, 50, and 48; (l′) SEQ ID NOs: 77, 60, and 48.

In some aspects, provided are isolated nucleic acid molecules or constructs or vectors comprising such nucleic acid molecules, wherein the nucleic acid molecules encode the fusion protein described herein.

In some embodiments, the nucleic acid molecule, construct, or vector comprises a nucleotide sequence selected from SEQ ID NOs: 1 to 20 or SEQ ID NOs: 25 to 36, or a sequence having at least 90% homology thereto and having the same biological activity.

In some aspects, provided is a cell, comprising the fusion protein, nucleic acid molecule, construct, or vector described herein.

In some aspects, provided is a composition, comprising the fusion protein, nucleic acid molecule, construct, vector, or cell described herein; and a carrier.

In some aspects, provided is the use of the fusion protein, nucleic acid molecule, construct, vector, cell, or composition described herein in the preparation of a medicament for immunotherapy.

In some embodiments, the medicament is used for positively regulating immune cell activity and/or enhancing immune response. In some embodiments, the medicament is used for the treatment of cancer, immunodeficiency disease, inflammatory disease, or infectious disease.

In some embodiments, the cancer is selected from the group consisting of bladder cancer, breast cancer, uterine cancer, endometrial cancer, ovarian cancer, colorectal cancer, colon cancer, head and neck cancer, lung cancer, stomach cancer, germ cell cancer, bone cancer, squamous cell carcinoma, skin cancer, central nervous system tumors, lymphoma, leukemia, sarcoma, virus-related cancers, small cell lung cancer, non-small cell lung cancer, gastrointestinal cancer, Hodgkin or non-Hodgkin lymphoma, pancreatic cancer, glioblastoma, glioma, cervical cancer, ovarian cancer, liver cancer, myeloma, salivary gland cancer, kidney cancer, basal cell carcinoma, melanoma, prostate cancer, vulvar cancer, thyroid cancer, testicular cancer, esophageal cancer, or head and neck cancer, and any combination thereof.

In some embodiments, the inflammatory or autoimmune disease is selected from the group consisting of type I diabetes, multiple sclerosis, rheumatoid arthritis, celiac disease, systemic lupus erythematosus, lupus nephritis, cutaneous lupus, idiopathic arthritis, Crohn's disease, ulcerative colitis or systemic sclerosis, graft-versus-host disease, psoriasis, alopecia areata, HCV-induced vasculitis, Sjögren's syndrome, pemphigus, ankylosing spondylitis, Behçet's disease, Wegener's granulomatosis, autoimmune hepatitis, sclerosing cholangitis, Goodpasture syndrome, and macrophage activation syndrome.

In some embodiments, the infectious disease is selected from infections caused by pathogenic viruses, bacteria, fungi, or parasites.

In some aspects, provided is a method for producing the fusion protein described herein, comprising: culturing the cell described herein under conditions suitable for expressing the fusion protein; and recovering the fusion protein.

Those skilled in the art may combine the aforementioned technical solutions and technical features in any combination without departing from the conception and the scope of protection of the application. Other aspects of the application are apparent to those skilled in the art from the disclosure herein.

### DESCRIPTION OF THE DRAWINGS

The application is further illustrated by the accompanying drawings, wherein these drawings are for illustrative purposes only and are not intended to limit the scope of the application.

FIG. 1: Schematic representation of the exemplary anti-TIGIT antibody HC-IL2 fusion protein structure of the application:

FIG. 1A: TIGIT antibody HC-IL2 fusion protein formed by connection to the N-terminus of the antibody;

FIG. 1B: IL2-TIGIT antibody HC fusion protein formed by connection to the C-terminus of the antibody.

FIG. 2: Flow cytometric analysis study showing the binding of anti-human TIGIT monoclonal antibodies to membrane-bound TIGIT.

FIG. 3: ELISA study showing the competitive binding of anti-human TIGIT monoclonal antibodies to CD155 and recombinant human TIGIT.

FIG. 4: The promotion effect of anti-human TIGIT monoclonal antibodies on IFNγ production by PHA-activated PBMCs.

FIG. 5: The effect of anti-human TIGIT humanized monoclonal antibodies on inhibiting tumor growth in mice in vivo:

FIG. 5A: Relationship between tumor volume and time for each mouse in each group;

FIG. 5B: Tumor weight for each mouse in each group at the end of the experiment (D17).

FIG. 6: SDS-PAGE identification of the prepared anti-TIGIT antibody HC-IL2 fusion protein.

FIG. 7: SEC-HPLC identification of the prepared anti-TIGIT antibody HC-IL2 fusion protein:

FIG. 7A: SEC-HPLC chromatogram of HC1-IL2 wt/LC1 (ART-Ig);

FIG. 7B: SEC-HPLC chromatogram of HC1-IL2mut/LC1 (ART-Ig);

FIG. 7C: SEC-HPLC chromatogram of IgG1 HC-IL2mut/LC (ART-Ig).

FIG. 8: BLI study of the binding affinity of the anti-TIGIT antibody HC-IL2 fusion protein to recombinant IL2Rα and IL2Rβ subunits:

FIG. 8A: Binding of HC1-IL2 wt/LC1 (ART-Ig) to IL2Ra;

FIG. 8B: Binding of HC1-IL2 wt/LC1 (ART-Ig) to IL2Rβ;

FIG. 8C: Binding of HC1-IL2mut/LC1 (ART-Ig) to IL2Ra;

FIG. 8D: Binding of HC1-IL2mut/LC1 (ART-Ig) to IL2RB.

FIG. 9: Flow cytometry study of the binding affinity of the anti-TIGIT antibody HC-IL2 fusion protein to membrane-bound IL2Rαγ and βγ subunits:

FIG. 9A: 293F cells co-transfected with IL2Rα and γ expressing plasmids or IL2Rβ and IL2Ry expressing plasmids, expressed IL2Rαγ dimers or IL2Rβγ dimers on the surface of 293F cells;

FIG. 9B: Binding of HC1-IL2 wt/LC1 (ART-Ig) and HC1-IL2mut/LC1 (ART-Ig) as well as control antibody IgG1 HC-IL2mut/LC (ART-Ig) to 293F cells expressing IL2Rαγ dimers and 293F cells expressing IL2Rβγ dimers.

FIG. 10: Study of the in vitro stimulation of lymphocyte CTLL-2 growth by the anti-TIGIT antibody HC-IL2.

FIG. 11: Study of the effect of the fusion protein on the growth of CD4+ and CD8+ lymphocytes in vivo:

FIG. 11A: The effect of the fusion protein on the growth of CD4+T lymphocytes in vivo;

FIG. 11B: The effect of the fusion protein on the growth of CD8+T lymphocytes in vivo.

FIG. 12: Study of the fusion protein's inhibition of tumor growth in vivo:

FIG. 12A: The effect of the fusion protein on tumor volume in vivo;

FIG. 12B: The effect of the fusion protein on tumor weight in vivo.

In the figures above, * indicates p<0.05, and ** indicates p<0.01.

### DETAILED DESCRIPTION

This disclosure provides a fusion protein of an anti-TIGIT antibody with IL-2 or its variants, comprising an anti-TIGIT antibody and an IL-2 molecule, or variants of one or both. Compared to the wild-type IL-2 molecule, the mutated interleukin-2 molecule can enhance affinity for IL-2Rβ or reduce affinity for IL-2Rα while enhancing affinity for IL-2RB. On the other hand, the TIGIT antibody-IL-2 fusion protein can prolong the half-life of IL-2, providing better biological activity than IL-2 alone; the TIGIT antibody component targets the fusion protein to the tumor environment, and generates a high concentration area around tumor cells, thus reducing side effects induced by IL-2. The TIGIT antibody can also release the tumor-killing activity of immune effector cells, synergistically kill tumor cells, and achieve a stronger inhibitory effect on tumors.

All numerical ranges provided herein are intended to clearly include all values between the endpoints and all ranges within those endpoints. Features mentioned in this application or in the embodiments mentioned can be combined. All features disclosed in this specification can be combined in any form of composition, and the individual features disclosed can be substituted with any alternative features that provide the same, equivalent, or similar purpose. Therefore, unless specifically stated otherwise, the disclosed features are only general examples of equivalent or similar features.

As used herein, “comprising”, “having”, or “including” encompasses “consisting of”, “consisting essentially of”, and “comprised of”; “consisting essentially of” and “consisted of” are sub-concepts of “comprising”, “having”, or “including”.

### Fusion Protein (Polypeptide)

As used herein, the term “fusion protein” refers to a fusion polypeptide molecule comprising an anti-TIGIT antibody polypeptide and an IL-2 polypeptide, wherein the components of the fusion protein are directly linked to each other via peptide bonds or connected through linkers. For clarity, the individual peptide chains of the antibody component of the fusion protein may be non-covalently linked, for example, through disulfide bonds. “Fusion” implies that the components are connected directly or through one or more peptide linkers by peptide bonds. As used herein, the terms “element” or “component” refer to amino acid sequences that constitute a part of the fusion protein. The terms “unit” or “monomer” refer to the basic fragments that make up the function of an element.

The term “antibody” as used herein is used in the broadest sense and encompasses various antibody structures including, but not limited to, monoclonal antibodies, polyclonal antibodies, multispecific antibodies (such as bispecific antibodies), and antibody fragments, as long as they exhibit the desired antigen-binding activity.

“Antibody fragments” refer to molecules other than the complete antibody, which comprise a portion of the complete antibody that binds to the antigen bound by the complete antibody. Examples of antibody fragments include, but are not limited to, Fv, Fab, Fab′, Fab′-SH, F(ab′)2, diabodies, linear antibodies, single-chain antibody molecules (e.g., scFv), and single-domain antibodies.

As used herein, the term “monoclonal antibody (mAb)” refers to an antibody obtained from a substantially homogeneous population, i.e., the individual antibodies comprised in the population are identical except for possibly naturally occurring mutations. Monoclonal antibodies are highly specific for a single antigenic site. Furthermore, unlike conventional polyclonal antibody preparations which typically include different antibodies directed against different determinants, each monoclonal antibody is directed against a single determinant on the antigen. In addition to their specificity, the advantage of monoclonal antibodies is that they are synthesized by hybridoma cultures without contamination by other immunoglobulins. The modifier “monoclonal” indicates the character of the antibody as being obtained from a substantially homogeneous population of antibodies, and this is not to be construed as requiring production of the antibody by any particular method.

Monoclonal antibodies and their fragments can be produced using various techniques known to those skilled in the art. For example, monoclonal antibodies can be made using the hybridoma method first described by Kohler et al., Nature, 256:495 (1975), or by recombinant DNA methods (U.S. Pat. No. 4,816,567). Monoclonal antibodies can also be isolated from phage antibody libraries using the techniques described in Clackson et al., Nature, 352:624-628 (1991) and Marks et al., J. Mol. Biol., 222:581-597 (1991).

As used herein, the term “antibody” or “immunoglobulin” refers to a glycoprotein with a molecular weight of approximately 150,000 daltons, composed of two identical light chains (L) and two identical heavy chains (H). Each light chain is connected to a heavy chain by one covalent disulfide bond, and the number of disulfide bonds between heavy chains of different immunoglobulin isotypes varies. Each heavy and light chain also comprises regularly spaced intrachain disulfide bonds. Each heavy chain has a variable region (VH) at one end, followed by several constant regions. Each light chain has a variable region (VL) at one end and a constant region at the other end; the constant region of the light chain corresponds to the first constant region of the heavy chain, and the variable region of the light chain corresponds to the variable region of the heavy chain. Specific amino acid residues form the interface between the variable regions of the light and heavy chains.

As used herein, the term “variable” refers to the fact that certain portions of the variable regions of antibodies are different in sequence, which forms the basis for the specificity of individual antibodies for their particular antigens. However, variability is not evenly distributed throughout the variable regions of antibodies. It is concentrated in three segments of the variable regions of both light and heavy chains known as complementarity-determining regions (CDRs) or hypervariable regions. The more conserved parts of the variable regions are known as the framework regions (FRs). The natural heavy and light chain variable regions each comprise four FRs, which are largely adopting a β-sheet configuration, linked by three CDRs, which in some cases form part of a β-sheet structure. The CDRs from each chain are brought together by the FRs and form the antigen-binding site of the antibody with the CDRs from another chain (see Kabat et al., NIH Publ. No. 91-3242, vol. I, pp. 647-669 (1991)). The constant regions do not participate directly in binding of the antibody to the antigen but exhibit various effector functions, such as participation in antibody-dependent cellular cytotoxicity.

The fusion protein disclosed herein comprises an anti-TIGIT antibody, including murine, chimeric, and humanized antibodies. The antibody comprises a heavy chain variable region (VH) comprising three complementarity-determining regions (CDRs) and a light chain variable region (VL) also comprising three CDRs. In some embodiments, non-human monoclonal antibodies can be humanized through the following methods: (1) homology replacement, wherein human framework regions (FRs) with high homology to the non-human counterparts are used for replacement; (2) surface reshaping, wherein the surface amino acid residues of non-human CDRs and FRs are reshaped to mimic the contours of human antibody CDRs or FR patterns; (3) compensation changes, changing amino acid residues at key positions to compensate for CDR grafting; (4) positional conservation, wherein humanization of monoclonal antibodies is carried out using the conserved sequences of FRs as templates, but key amino acid residues from the variable region of the non-human monoclonal antibody is retained.

The disclosure encompasses the use of complete monoclonal antibodies as well as their immunologically active antibody fragments (antigen-binding fragments), such as Fab, Fab′, F(ab′)2, Fd, single-chain Fv or scFv, disulfide-linked Fv, V-NAR domains, IgNar, intrabodies, IgGΔCH2, mini-antibodies, F(ab′) 3, tetrabodies, tribodies, bispecific antibodies, single-domain antibodies, DVD-Ig, Fcab, mAb2, (scFv)2 or scFv-Fc, among others.

As used herein, the term “sequence identity” or “% identity” refers to the percentage of identical residues (e.g., amino acids or nucleotides) in a candidate sequence compared to a reference sequence after aligning the sequences and (if necessary) introducing gaps to achieve the highest percentage of sequence identity. For instance, as used herein, “at least 70% sequence identity” means the sequence identity between the candidate sequence and the reference sequence is at least 70%, including 75, 80, 85, 90, 91, 92, 93, 94, 95, 96, 97, 98, 99, 100%, or any point in between.

The term “Fc region” as used herein refers to the C-terminal region in the antibody heavy chain that comprises at least a part of the constant region. This term includes natural sequence Fc regions and variant Fc regions. The IgG Fc region comprises IgG CH2 and IgG CH3 domains, both of which can be of natural sequence or comprise mutations. Techniques such as “knob-in-hole” (KiH) (Merchant et al (1998) Nat Biotech 16, 677-681) or electrostatic steering interaction-based techniques (ART-Ig, see for example PCT/JP2006/306803) can be used to maximally produce heterodimeric antibodies. The KiH technique involves introducing mutations in the area wherein two heavy chain Fcs bind tightly together, wherein one heavy chain introduces mutations that form a knob (e.g., S354C and T366W, EU numbering), and the other heavy chain introduces mutations that form a hole (e.g., Y349C, T366S, L368A, Y407V, EU numbering) to produce a structurally more stable heterodimeric antibody. The ART-Ig technique involves introducing positively and negatively charged mutations in the area wherein two heavy chain Fcs bind tightly together, wherein one heavy chain introduces positive charge amino acid mutations (ART-Ig-P) (e.g., E356K and H435R, EU numbering), and the other heavy chain introduces negative charge amino acid mutations (ART-Ig-N) (e.g., K439E, EU numbering) to produce a structurally more stable heterodimeric antibody. In this disclosure, IL2 can be fused to either heavy chain. For example, IL2 can be fused to the antibody heavy chain HC (hole) or HC (ART-Ig-P).

The anti-TIGIT antibodies in the fusion protein described herein can be obtained by methods known in the field or be known or obtained antibodies to TIGIT. The anti-TIGIT antibodies used in the fusion protein of this disclosure can include one or more of the following characteristics:
(i) Specifically binds to human TIGIT in vitro or in vivo, e.g., its binding affinity to human TIGIT as determined by ELISA with an EC50 of 0.001 nM to 100 nM, 0.01 nM to 50 nM, 0.1 nM to 10 nM;
(ii) Competitively inhibits the binding of CD155 to TIGIT, e.g., its specific blockade of CD155 to human TIGIT as determined by ELISA with an IC50 of 0.005 μg/ml to 1 μg/ml, 0.01 μg/ml to 0.9 μg/ml;
(iii) Binds to human or primate TIGIT but not mouse TIGIT;
(iv) Positively regulates the activity of immune cells (e.g., T cells and NK cells), e.g., enhancing the production of pro-immune response cytokines (e.g., IFNγ) by human peripheral blood mononuclear cells (PBMCs) (e.g., lymphocytes, T cells), reducing or reversing NK cell exhaustion;
(v) Reduces or eliminates cells expressing TIGIT, e.g., Treg cells, reducing immune suppression;
(vi) Enhances immune response; and
(vii) Has a prophylactic and/or therapeutic effect against tumors, infections, or infectious diseases.

In some embodiments, the anti-TIGIT antibody or antigen-binding fragment thereof in the fusion protein comprises heavy chain complementarity-determining regions (VH CDRs) 1 to 3 and light chain complementarity-determining regions (VL CDRs) 1 to 3 selected from the group consisting of:
VH CDR1 selected from the group consisting of: SEQ ID NO: 81 or 89;
VH CDR2 selected from the group consisting of: SEQ ID NO: 82 or 90;
VH CDR3 selected from the group consisting of: SEQ ID NO: 83 or 91;
VL CDR1 selected from the group consisting of: SEQ ID NO: 85 or 93;
VL CDR2 selected from the group consisting of: SEQ ID NO: 86 or 94; and
VL CDR3 selected from the group consisting of: SEQ ID NO: 87 or 95, or
a sequence having at least 80%, 85%, 90%, 95%, 98%, 99% sequence identity thereto.

In some embodiments, the anti-TIGIT antibody or antigen-binding fragment thereof in the fusion protein comprises amino acid sequences of VH CDRs 1 to 3 as follows: SEQ ID NOs: 81, 82, and 83; or SEQ ID NOs: 89, 90, and 91; and amino acid sequences of VL CDRs 1 to 3 as follows: SEQ ID NOs: 85, 86, and 87; or SEQ ID NOs: 93, 94, and 95; or a sequence having at least 80%, 85%, 90%, 95%, 98%, 99% sequence identity thereto.

In some embodiments, the anti-TIGIT antibody or antigen-binding fragment thereof in the fusion protein comprises VH CDRs 1 to 3 and VL CDRs 1 to 3 selected from the group consisting of: SEQ ID NOs: 81, 82, and 83 and SEQ ID NOs: 85, 86, and 87; or SEQ ID NOs: 89, 90, and 91 and SEQ ID NOs: 93, 94, and 95; or sequences having at least 80%, 85%, 90%, 95%, 98%, 99% sequence identity thereto.

In some embodiments, the anti-TIGIT antibody or antigen-binding fragment thereof in the fusion protein comprises a heavy chain variable region VH selected from SEQ ID NO: 80 or 88; and/or a light chain variable region VL selected from SEQ ID NO: 84 or 92; or sequences having at least 80%, 85%, 90%, 95%, 98%, 99% sequence identity thereto.

In some embodiments, the anti-TIGIT antibody or antigen-binding fragment thereof comprises a combination of VH and VL selected from: VH of SEQ ID NO: 80 and VL of SEQ ID NO: 84; or VH of SEQ ID NO: 88 and VL of SEQ ID NO: 92; or sequences having at least 80%, 85%, 90%, 95%, 98%, 99% sequence identity thereto.

“Natural IL-2”, also known as “wild-type IL-2” (wtIL2), refers to the naturally occurring IL-2, as opposed to “modified IL-2”, also known as “mutant IL-2” (mutIL2), which has mutations or modifications relative to the naturally occurring IL-2, for example, to change one or more properties of natural IL-2 such as stability, activity, etc. Modified IL-2 molecules may include modifications within the amino acid sequence, such as amino acid substitutions, deletions, or insertions.

In some embodiments, mutant IL-2 molecules differ in the IL-2 regions that bind to the IL-2Rα subunit and the IL-2Rβ subunit. Introducing amino acid mutations into these two regions of IL-2 can alter the affinity of IL-2 for binding to the IL-2Rα subunit and the IL-2Rβ subunit. For example, IL-2 mutants (L80F, R81D, L85V, 186V, 192F) have enhanced affinity for binding to the IL-2Rβ subunit (Levin et al. (2012) Nature 484, 529-533), while the IL-2 mutant (F42A) has reduced affinity for binding to the IL-2Rα subunit. Synthetic genes for IL-2 mutants (L80F, R81D, L85V, 186V, 192F) can be generated, and then site-directed mutagenesis can be used to introduce the amino acid mutation (F42A) into this gene sequence, thereby constructing an IL-2mut gene encoding a mutant IL-2 with reduced affinity for binding to the IL-2Rα subunit and enhanced affinity for binding to the IL-2Rβ subunit.

As mentioned in the background, the structure and function of IL-2 are already somewhat researched and understood in the field. In this application, IL-2 polypeptides known in the field can be used, as well as their natural variants and fragments (such as splice variants or allelic variants), and non-natural variants with IL-2 activity.

The wild-type human IL-2 polypeptide used herein may comprise the amino acid sequence of residues 21-153 (Accession No. UniProtKB-P60568) SEQ ID NO: 96 (human IL-2) or be encoded by a nucleic acid molecule comprising SEQ ID NO: 97, or homologous sequences with the same or similar activity (which can be obtained through databases or alignment softwares known in the field).

The mutant IL-2 polypeptides of this disclosure can be selected from: (a) polypeptides with one or more additional amino acid residues in two regions of the aforementioned wild-type IL-2 amino acid sequence; or (b) proteins or polypeptides derived from (a) that have been substituted, deleted, or added with one or several amino acids within the wild-type IL-2 sequence and possess wild-type IL-2 activity. For example, the mutant IL-2 polypeptides of this disclosure may include the aforementioned mutations that reduce the affinity for binding to the IL-2Rα subunit and/or enhance the affinity for binding to the IL-2Rβ subunit. In some embodiments, the sequence of the mutant IL-2 polypeptide may be as shown in SEQ ID NO: 98 or be encoded by a nucleic acid molecule comprising SEQ ID NO: 99.

One or more polypeptide components in the fusion protein disclosed herein are preferably encoded by human genes or their homologs or family genes or are humanized. Variants of proteins or polypeptides in this disclosure include (but are not limited to): one or more (typically 1-50, preferably 1-30, more preferably 1-20, most preferably 1-10, e.g., 1, 2, 3, 4, 5, 6, 7, 8, 9, or 10) amino acid deletions, insertions, and/or substitutions, and the addition of one or a few (typically within 20, preferably within 10, more preferably within 5) amino acids at the C-terminus and/or N-terminus. For instance, in the field, substituting amino acids with functionally similar or analogous amino acids usually does not change the function of the protein or polypeptide. Similarly, adding one or a few amino acids at the C-terminus and/or N-terminus also usually does not change the function of the protein or polypeptide, for example, a fusion protein may or may not include the initiating methionine residue and still retain its desired antiviral activity.

Variants of polypeptides include homologous sequences, conservative variants, allelic variants, natural mutants, induced mutants, and proteins encoded by sequences capable of hybridizing under high or low stringency conditions with their protein encoding sequence. Depending on the host used for recombinant production, the proteins or polypeptides of the invention can be glycosylated or non-glycosylated.

In the fusion protein of the invention, the components (anti-TIGIT antibody and IL-2 molecule) are genetically fused to each other. The fusion protein can be designed such that its components are directly fused to each other or indirectly fused via a linker sequence. The composition and length of the linker can be determined according to methods well known in the field and can be tested for efficacy. Additional sequences may also be included to incorporate cleavage sites to separate the individual components of the fusion protein (if desired), such as endopeptidase recognition sequences.

The polypeptide components or peptide units within the components in the fusion protein of this disclosure can be connected through a linker. In this application, flexible linkers are preferred to allow for interactions between polypeptide components or peptide units. Linkers that can be used in the fusion protein of this application may comprise 2-300 amino acid residues, for example, 5-100, 10⁻⁵⁰, or 15-30 amino acid residues. Exemplary linkers can be glycine linkers, such as (G)ₙ, or glycine/serine linkers, such as amino acid sequences of (GS)ₙ, (GGS)ₙ, (GGGS)ₙ, (GGGGS)ₙ, or (GGGGGS)ₙ, wherein n is an integer of 1, 2, 3, 4, 5, 6, 7, 8, 9, or 10.

The fusion protein of this disclosure may also include a signal peptide, such as an amino acid sequence that functions to guide the secretion, localization, and/or transport of the fusion protein, typically ranging from 5-50 amino acids in length. In some embodiments, the signal peptide can be selected from, for example, the tPA2 signal peptide, TFF2 signal peptide, IL-2 signal peptide, bPRL signal peptide, CD33 protein signal peptide, among others.

Furthermore, the fusion protein of this disclosure can include a tag, for example, a tag for purification, detection, or localization, such as fluorescent markers, non-radioactive isotope markers, biotin-related markers, phosphorylation modification tags, peptide tags.

### Encoding Nucleic Acid Molecules, Vectors, Host Cells

The fusion protein of the invention can be obtained, for example, by solid-phase peptide synthesis (such as Merrifield solid-phase synthesis) or through recombinant production. For recombinant production, one or more polynucleotides encoding the fusion protein (or fragments thereof) are isolated and inserted into one or more vectors for further cloning and/or expression in host cells. Such polynucleotides can be easily isolated and sequenced using conventional procedures.

In some embodiments, vectors (preferably expression vectors) comprising one or more nucleic acid molecules encoding the fusion protein or its components are provided. Expression vectors comprising the coding sequence for the fusion protein (or its fragments) along with suitable transcription/translation control signals can be constructed using methods known to those skilled in the art. These methods include in vitro recombinant DNA techniques, synthetic techniques, and in vivo recombination/genetic recombination.

The expression vector can be a plasmid, a part of a virus, or a nucleic acid fragment. The expression vector comprises an expression cassette, wherein a polynucleotide encoding the fusion protein (fragment) (i.e., the coding region) is cloned in operable association with a promoter and/or other transcription or translation control elements. As used herein, the “coding region” refers to a part of the nucleic acid composed of codons that are translated into amino acids. Two or more coding regions may be present in a single polynucleotide construct (e.g., on a single vector) or may be present in separate polynucleotide constructs, e.g., on separate (different) vectors. Moreover, any vector may comprise a single coding region or may comprise two or more coding regions, for example, vectors of the invention may encode one or more polypeptides which are separated into final proteins post-translationally or co-translationally via proteolytic cleavage. Additionally, vectors, polynucleotides, or nucleic acids of the invention may encode heterologous coding regions, which may be fused to polynucleotides encoding the fusion protein (fragment) or its variants or derivatives of the invention or not. Heterologous coding regions include but are not limited to specialized elements or motifs such as secretory signal peptides or heterologous functional domains. An operable association exists when a gene product, such as the coding region of a peptide, is linked in some way to a regulatory sequence that influences or controls the expression of the gene product.

The polynucleotides and nucleic acid coding regions of the invention may be combined with another coding region encoding a secretory or signal peptide, which directs the secretion of the peptide encoded by the polynucleotides of the invention. For example, if it is desired to secrete the fusion protein, DNA encoding a signal sequence may be positioned upstream of the nucleic acid encoding the fusion protein or its fragment of the invention. It is known to those skilled in the art that polypeptides secreted by vertebrate cells typically have a signal peptide fused to the N-terminus of the polypeptide, which is cleaved off to generate the secreted or “mature” form of the peptide. In certain embodiments, the natural signal peptide, such as the immunoglobulin heavy or light chain signal peptide, or a functional derivative of the sequence that retains the ability to guide secretion of the peptide operably linked thereto, is used. Alternatively, heterologous mammalian signal peptides or their functional derivatives can be used. For example, the wild-type leader sequence can be replaced with the leader sequence of human tissue plasminogen activator (TPA) or mouse beta-glucuronidase.

In some embodiments, host cells comprising one or more of the polynucleotides or vectors of the invention are provided. As used herein, the term “host cell” refers to any cell type capable of being engineered to produce the fusion protein or its fragment of the invention. Host cells suitable for replication and supporting the expression of the fusion protein are known in the art. When appropriate, such cells can be transfected or transduced with specific expression vectors and can be cultured in large quantities in bioreactors for seeding large-scale fermenters to obtain enough fusion protein for clinical applications.

Suitable host cells include prokaryotic microorganisms such as Escherichia coli, or various eukaryotic cells, such as Chinese hamster ovary cells (CHO), insect cells, etc. Examples of available mammalian host cell lines include but are not limited to: Chinese hamster ovary (CHO) cells, monkey kidney CVI cell line transformed with SV40 (COS-7), human embryonic kidney line (293 or 293T cells), baby hamster kidney cells (BHK), mouse sertoli cells (TM4 cells), African green monkey kidney cells (VERO-76), human cervical carcinoma cells (HELA), macaque kidney cells (MDCK), bovine mammary cells (BRL 3A), human lung cells (W138), human liver cells (Hep G2), mouse breast tumor cells (MMT 060562), TRI cells, MRC 5 cells, FS 4 cells, and myeloma cell lines such as YO, NS0, P3X63, and Sp2/0.

Host cells include not only cultured cells, such as mammalian culture cells, yeast cells, insect cells, bacterial cells, and plant cells, but also cells comprised in transgenic animals, transgenic plants, or cultured plant or animal tissues.

In one embodiment, a method for producing the fusion protein of the invention is provided, wherein the method comprises culturing host cells comprising the polynucleotides encoding the fusion protein under conditions suitable for the expression of the fusion protein and recovering the fusion protein from the host cells (or the culture medium of the host cells).

The recombinant fusion proteins of the application can be expressed intracellularly, on the cell membrane, or secreted extracellularly. If necessary, the recombinant protein can be separated and purified using various separation methods based on its physical, chemical, and other properties. These methods are well known to those skilled in the art. Examples of these methods include but are not limited to conventional renaturation processes, treatment with protein precipitants (salting out methods), centrifugation, osmotic lysis, ultrafiltration, ultracentrifugation, size-exclusion chromatography (gel filtration), adsorption chromatography, ion exchange chromatography, high-performance liquid chromatography (HPLC), and other various liquid chromatography techniques, as well as combinations of these methods.

### Products Such as Compositions or Medicaments

Also provided herein is a product comprising an effective amount of the fusion protein, a vector comprising the encoding molecule for the fusion protein, host cells, or a composition described herein, as well as a pharmaceutically or physiologically acceptable carrier. As used herein, the term “active ingredient” refers to the fusion protein described herein, its encoding nucleic acid molecule, the construct or vector comprising the nucleic acid molecule, host cells, or a aforementioned composition.

In preferred embodiments, the product described herein can be used in use for positively regulating immune cell activity and/or enhancing immune response; and/or for the treatment of cancer, immunodeficiency diseases, inflammatory diseases, or infectious diseases. As used herein, the terms “comprising” or “including” encompass “comprising”, “consisting essentially of”, and “consisting of”. As used herein, the term “pharmaceutically acceptable” refers to substances that are suitable for use in humans and/or animals without causing excessive adverse side effects (such as toxicity, irritation, and allergic reactions), that is, they have a reasonable benefit/risk ratio. As used herein, the term “effective amount” refers to a quantity capable of producing a function or activity in humans and/or animals and can be accepted by humans and/or animals.

As used herein, the term “pharmaceutically acceptable carrier” refers to a carrier for administering a therapeutic agent, including various excipients and diluents. This term denotes such pharmaceutically carrier agents that are not active ingredients themselves and do not exhibit excessive toxicity upon administration. Suitable carriers are well known to those skilled in the art. A full discussion of pharmaceutically acceptable excipients can be found in “Remington's Pharmaceutical Sciences” (Mack Pub. Co., N.J., 1991).

In the composition, the pharmaceutically acceptable carrier may comprise liquids such as water, saline, glycerol, and ethanol. Additionally, these carriers may also comprise auxiliary substances such as fillers, disintegrants, lubricants, flow agents, effervescing agents, wetting or emulsifying agents, sweeteners, pH buffering agents, etc. Typically, these substances can be formulated in non-toxic, inert, and pharmaceutically acceptable aqueous media, wherein the pH is generally about 5 to 8, preferably about 6 to 8.

As used herein, the term “unit dosage form” refers to a dosage form prepared for easy administration in a single dose required for each application, including but not limited to various solid forms (such as tablets, freeze-dried powders), liquid forms (such as solutions), capsule forms, and controlled-release forms.

In another preferred embodiment of the invention, the composition is in unit dosage form or in a multi-dose form, and the content of the active ingredient therein ranges from 0.01 to 2000 mg per dose, preferably 0.1 to 1500 mg per dose, more preferably 1 to 1000 mg per dose. In another preferred example of the invention, 1 to 6 doses of the composition of the invention are administered per day, preferably 1 to 3 doses; most preferably, the daily dosage is 1 dose.

The pharmaceutical composition of the invention can be formulated into various dosage forms as needed and the beneficial dosage for patients can be determined by physicians based on factors such as the type of patient, age, weight, general health condition, mode of administration, etc. The form of the pharmaceutical composition of the invention can be suitable for the desired mode of administration, for example, intravenous, intradermal, intraarterial, intraperitoneal, intralesional, intracranial, intraarticular, intraprostatic, intrasplenic, intrarenal, intrapleural, intratracheal, intranasal, intravitreal, intravaginal, rectal, intratumoral, intramuscular, intraperitoneal, subcutaneous, subconjunctival, intravesical, mucosal, intrapericardial, umbilical, intraocular, oral, topical, inhalation (such as aerosol inhalation), injection, infusion, continuous infusion, local perfusion directly bathing the target cells, via catheter, via lavage, in emulsion, in liquid composition (such as liposomes), or by other methods or any combination of the foregoing.

To enhance therapeutic effects, the active ingredients or products of the invention can be used in combination with each other and also in conjunction with other medications and treatment methods, such as those used for the prevention and treatment of cancer. For example, if the fusion protein of the invention is used for the prevention and/or treatment of cancer, other drugs or methods clinically used for cancer treatment can be used simultaneously or sequentially, including but not limited to: chemotherapy drugs, radiotherapy, surgery, etc.

Cancers that can be treated with the fusion protein described herein include but are not limited to: bladder cancer, breast cancer, uterine cancer, endometrial cancer, ovarian cancer, colorectal cancer, colon cancer, head and neck cancer, lung cancer, stomach cancer, germ cell tumor, bone cancer, squamous cell carcinoma, skin cancer, central nervous system tumors, lymphoma, leukemia, sarcoma, virus-related cancers, small cell lung cancer, non-small cell lung cancer, gastrointestinal cancer, Hodgkin or non-Hodgkin lymphoma, pancreatic cancer, glioblastoma, glioma, cervical cancer, ovarian cancer, liver cancer, myeloma, salivary gland cancer, kidney cancer, basal cell carcinoma, melanoma, prostate cancer, vulvar cancer, thyroid cancer, testicular cancer, esophageal cancer, or head and neck cancer, and any combination thereof.

Inflammatory diseases or autoimmune diseases that can be treated with the fusion protein described herein include but are not limited to: Type 1 diabetes, multiple sclerosis, rheumatoid arthritis, celiac disease, systemic lupus erythematosus, lupus nephritis, cutaneous lupus, idiopathic arthritis, Crohn's disease, ulcerative colitis or systemic sclerosis, graft-versus-host disease, psoriasis, alopecia areata, HCV-induced vasculitis, Sjögren's syndrome, pemphigus, ankylosing spondylitis, Behçet's disease, Wegener's granulomatosis, autoimmune hepatitis, sclerosing cholangitis, Guillian-Barre syndrome, and macrophage activation syndrome.

Infectious diseases that can be treated with the fusion protein herein include but are not limited to infections caused by pathogenic viruses, bacteria, fungi, or parasites.

### EXAMPLES

In conjunction with specific examples, this application is further elucidated below. It should be understood that these examples are provided for illustration only and are not intended to limit the scope of this application. Those skilled in the art may make appropriate modifications and variations within the scope of this application.

Experimental methods in the following examples, wherein specific conditions are not mentioned, can adopt conventional methods in the field, such as those referenced in “Molecular Cloning: A Laboratory Manual” (3rd edition, Cold Spring Harbor Laboratory Press, New York, 1989) or according to conditions recommended by suppliers. DNA sequencing methods are conventional in the field and can also be provided by commercial companies.

Unless otherwise specified, percentages and parts are calculated by weight. All technical and scientific terms used herein have the same meaning as commonly understood by one of ordinary skill in the art, unless defined otherwise. Any similar or equivalent methods and materials to those described herein can be applied in the methods of this application. The preferred embodiments and materials described herein are for illustrative purposes only.

### Example 1. Generation of Anti-TIGIT Monoclonal Antibodies and their Encoding Sequences, Performance Evaluation, and Construction of Anti-TIGIT Antibody-IL2 Fusion Protein Gene

1.1 Generation of Anti-TIGIT Monoclonal Antibodies and their Encoding Sequences

The anti-TIGIT antibodies are part of several humanized monoclonal antibodies against TIGIT invented by the applicant, with their preparation method and amino acid sequences disclosed in the Chinese patent application CN202111105544.0 titled “Monoclonal Antibody Targeting TIGIT”, filed on Sep. 22, 2021.

Specifically, by conventional methods, Balb/c mice (aged 6-8 weeks, weighing about 18 g, female, obtained from the Experimental Animal Division of the Institute of Planned Parenthood Research) were immunized with intraperitoneal and subcutaneous injections of recombinant human TIGIT (Human TIGIT His, Sino Biological, 10917-H08H Accession #NP 776160.2.Met1-Phe138, expressed with a C-terminal histidine tag). Spleen cells from immunized mice were harvested. These spleen cells were fused with mouse myeloma cells (obtained from the Cell Bank of the Chinese Academy of Sciences, catalog number TCM18), and positive clones were screened using TIGIT-Fc (Human TIGIT (Fc Tag), Sino Biological, 10917-H02H) via ELISA, resulting in several monoclonal clones, among which clones 7103-01 and 7103-07 both exhibited IgG1 heavy chains and Kappa light chains.

The resulting cell lines were cultured and expanded. Supernatants were collected and the fusion proteins were purified using Protein-A affinity chromatography columns. Gel analysis was performed, loading 2 μg of reduced and non-reduced samples in each lane, at 150 V for 1 hour. The molecular weight of the non-reduced antibody was around 150 KD, and the molecular weights of the reduced antibody were around 50 KD and 25 KD, consistent with the molecular characteristics of antibodies. The binding affinity of the obtained monoclonal antibodies to recombinant human TIGIT antigen (Human TIGIT (His), Sino Biological, 10917-H08H) was detected via ELISA, EC₅₀ values for the binding affinity of clones 7103-01 and 7103-07 to TIGIT antigen were shown to be 0.0647 nM and 0.075 nM, respectively.

Total RNA was extracted from the hybridoma cells for reverse transcription. The VH and VL genes of the hybridoma antibody were amplified from the obtained cDNA and sequenced to obtain the sequences for the VH and VL sequences of the TIGIT monoclonal antibody. Before antibody humanization, recombinant human-mouse chimeric antibody expression plasmids were constructed by building the V region of the hybridoma antibody onto the chimeric antibody expression vector (pcDNA3.1), linking the V region sequences to expression vectors with constant regions of human IgG1 or human λ/κ, and transiently expressing and purifying human-mouse chimeric antibodies in HEK293F cells. Then, based on the sequences of the mouse parental antibodies of clones 7103-01 and 7103-07, humanized antibody sequences were designed. The steps were as follows: Firstly, three-dimensional molecular models of the variable regions were constructed using homology modeling methods with Discovery Studio and Schrödinger Antibody Modeling. Subsequently, the structures of the parental antibody variable regions and CDRs were simulated by comparing with existing antibody structures in databases. At the same time, human germline sequences highly homologous to the VH and VL of the mouse parental antibody were selected for comparison. For the heavy chain VH, IGHV1, which had the highest homology, was chosen as the template for humanization design. For the light chain VL, IGKV1 was chosen as the template for humanization design. The resulting humanized anti-TIGIT monoclonal antibodies comprised the following combinations of heavy and light chain sequences:

TABLE 1
Combinations of different light and heavy chains of
humanized antibodies and their corresponding numbers

| Source of | | |
|---|---|---|
| Humanized | Combination of Light and Heavy | Antibody |
| Antibody | Chains of Humanized Antibody | Number |
| 7103-01 | huVH4VL1 | P03489 |
| 7103-07 | huVH2VL1 | P03479 |

TABLE 2
P03489 heavy chain and light chain sequences

| | | SEQ ID | |
|---|---|---|---|
| Description | | NO | Sequence |
| P03489 | Full | 80 | QVQLVQSGAEVKKPGASVKVSCKASGYTFISYNIY |
| 7103-01 | length | | WVRQAPGQGLEWMGGVNPSNGNTNFNENFQGRV |
| huVH4 | sequence | | TMTVDTSISTAYMELSRLRSDDTAVYYCTRGNYY |
| | | | GYEFAYWGQGTTVTVSS |
| | CDR1 | 81 | SYNIY |
| | CDR2 | 82 | GVNPSNGNTNFNENFQG |
| | CDR3 | 83 | GNYYGYEFAY |
| P03489 | Full | 84 | DIQMTQSPSSMSASVGDRVTITCKASQHVSTAVAW |
| 7103-01 | length | | YQQKPGKAPKLLIYSPSYRYTGVPSRFSGSGSGTDF |
| huVL1 | sequence | | TFTISSVQPEDIATYYCQQHYSTPWTFGGGTKLEIK |
| | CDR1 | 85 | KASQHVSTAVA |
| | CDR2 | 86 | SPSYRYT |
| | CDR3 | 87 | QQHYSTPWT |

TABLE 3
P03479 heavy chain and light chain sequences

| | | SEQ ID | |
|---|---|---|---|
| Description | | NO | Sequence |
| P03479 | Full | 88 | EVQVVESGGGLVKPGGSLRLSCAASGFTFSTYAMS |
| 7103-07 | length | | WVRQAPGKGLEWVAEISSGGSHTFYADTVKGRFTI |
| huVH2 | sequence | | SRDNAKNTLYLQMNSLRAEDTAVYYCARKTLDY |
| | | | YALDYWGQGTTVTVSS |
| | CDR1 | 89 | TYAMS |
| | CDR2 | 90 | EISSGGSHTFYADTVKG |
| | CDR3 | 91 | KTLDYYALDY |
| P03479 | Full | 92 | DVQITQSPSYLSASVGDRVTINCRASKSISKYLAWY |
| 7103-07 | length | | QQKPGKAPKLLIYSGSRLQSGIPSRFSGSGYGTDFT |
| huVL1 | sequence | | LTISSLQPEDFATYYCQQHNEYPWTFGGGTKLEIK |
| | CDR1 | 93 | RASKSISKYLA |
| | CDR2 | 94 | SGSRLQS |
| | CDR3 | 95 | QQHNEYPWT |

Based on the humanization design results described above, the sequences were constructed into the pcDNA3.4 vector, followed by PCR, restriction digestion, ligation, transformation, identification, sequencing, comparison, and plasmid extraction to obtain the plasmid. Using the ExpiCHO-S expression system (Thermo Fisher, A29133) and according to the expression combinations after humanization design, the aforementioned plasmids were expressed in ExpiCHO-S cells for a period of 7 days, and on the 6th day of expression, the expression levels were measured by HPLC. Finally, antibody proteins were obtained through a further purification using Protein A affinity chromatography.

To determine whether the humanized TIGIT monoclonal antibodies could bind to recombinant human TIGIT protein, an ELISA test was conducted. The results showed that the humanized monoclonal antibodies against human TIGIT, P03489 and P03479, were able to bind to human TIGIT protein with EC50 values of 0.5 nM and 0.09 nM, respectively.

### 1.2 Study of Antibody Binding Activity to TIGIT Using Bio-Layer Interferometry (BLI) Technology

The GATOR (ProbeLife) detection apparatus with HFC (LOT #2007065 Tray 2), and Human antibody Capture probes were used to measure the dissociation constant (Kd) of the binding between human-mouse chimeric TIGIT monoclonal antibodies (7103-01 (hFc) and 7103-07 (hFc)) and humanized TIGIT monoclonal antibodies with recombinant human TIGIT. The monoclonal antibodies were diluted to 30 nM in binding buffer (Q buffer [PBS (10 mM PH7.4)+0.02% Tween 20+0.2% BSA]). Recombinant human TIGIT protein was diluted in Q buffer by a 2-fold gradient to different concentrations, varying in the range of 40 nM to 10 nM. The kinetic association measurements were initiated by placing the antibody capture sensors into the above serially diluted antigen solutions.

Table 4 shows the BLI detection results. The humanized antibody #P03489 of 7103-01 showed a binding affinity to TIGIT with a Kd comparable to its parental human-mouse chimeric antibody 7103-01 (hFc), with Kd values of 1.19×10⁻⁹ M and 1.73×10⁻⁹ M, respectively. The humanized antibody #P03479 of 7103-07 showed a weaker binding affinity to TIGIT compared to its parental human-mouse chimeric antibody 7103-07 (hFc), with Kd values of 8.43×10⁻⁹ M and 2.96×10⁻⁹ M, respectively.

TABLE 4
Summary of binding kinetics constants for TIGIT mAbs with recombinant
human TIGIT

| | | TIGIT-His | | | | |
|---|---|---|---|---|---|---|
| TIGIT mAbs | Sample ID | K_{D} (M) | ka (1/Ms) | kd (1/s) | R² | Rₘₐₓ (nm) |
| 7103-01 (hFc) | 7103-01 (hFc) | 1.73E−09 | 5.49E+05 | 9.51E−04 | 0.992 | 0.293 |
| 7103-01-huVH4VH1 | P03489 | 1.19E−09 | 4.89E+05 | 5.84E−04 | 0.993 | 0.289 |
| 7103-07 (hFc) | 7103-07 (hFc) | 2.96E−09 | 5.73E+05 | 1.69E−03 | 0.985 | 0.311 |
| 7103-07-huVH2VH1 | P03479 | 8.43E−09 | 5.87E+05 | 4.95E−03 | 0.992 | 0.252 |

### 1.3 Study of the Binding of Human-Mouse Chimeric and Humanized Monoclonal Antibodies to TIGIT Protein on the Cell Membrane Using Flow Cytometry

A stable CHO-K cell line expressing full-length human TIGIT (CHO-K-TIGIT) was established as follows: A plasmid comprising the full-length human TIGIT cDNA (NM_173799.2), purchased from Sino Biological, was used to clone the cDNA into a mammalian expression vector, pcDNA3.1 (Invitrogen), which comprises the gene for rat glutamine synthetase. The constructed plasmid was then transfected into CHO-K cells using electroporation (Bio-Rad, Gene Pulser Xcell). After 24-48 hours of culture of the transfected cells in OptiCHO medium (Invitrogen), the medium was switched to selection medium. The selection medium comprised OptiCHO, 5 μg/ml recombinant human insulin, and 10 μM methionine sulfoximine (MSX). The cells were cultured at 37° C. in an incubator with 8% CO₂. After three weeks, flow cytometry sorting (FACS) was performed to screen cultured cells using anti-TIGIT antibodies to obtain CHO-K monoclonal cell lines expressing human TIGIT on the cell membrane.

The flow cytometry method to study the binding of TIGIT antibodies to TIGIT protein on the cell membrane is briefly described as follows: Firstly, FACS buffer was prepared, which was a 1×PBS solution with 0.5% BSA. The required number of CHO-K-TIGIT cells, approximately 1×10⁵ to 5×10⁵ cells per sample, was taken and suspended in FACS buffer. TIGIT mAbs (7103-01 (hFc), 7103-07 (hFc), P03479, and P03489) and the control antibody Tiragolumab (Roche, in phase III clinical trials; used as a positive control sample) were diluted to a certain ratio in FACS buffer to resuspend the cells, with a volume of about 100 μL. The cells were incubated in a refrigerator at 4° C. for 30 minutes, washed once with FACS buffer, and then resuspended in FITC-conjugated anti-human IgG (Abcam: 6854) diluted in FACS buffer. The cells were incubated again in a refrigerator at 4° C. for 30 minutes. Finally, after washing the cells with FACS buffer, the cells were resuspended in 200 μL of FACS buffer for analysis, and subjected to detection.

FIG. 2 shows the results of the flow cytometry study. As shown in the figure, the humanized TIGIT monoclonal antibody P03479 bound well to CHO-TIGIT, with an EC50 of 0.144 μg/mL (0.963 nM) for binding on the cell membrane of CHO-TIGIT cells. Compared to the parental antibody (7103-07 (hFc)) with EC₅₀ of 0.201 μg/mL (1.347 nM) and Tiragolumab with EC₅₀ of 0.574 μg/mL (3.844 nM), P03479 exhibited higher affinity.

### 1.4 Inhibition of Recombinant Human TIGIT-CD155 Binding by Human-Mouse Chimeric and Humanized TIGIT Monoclonal Antibodies

To determine whether human-mouse chimeric and humanized TIGIT monoclonal antibodies can inhibit the binding between CD155 and TIGIT, an in vitro ELISA assay was conducted. Tiragolumab was used as a positive control. The antigen TIGIT (Human TIGIT (His), Sino Biological, 10917-H08H) was diluted to 1 μg/mL with NaHCO₃ solution at pH=9.6, and 50 μL per well was added to a 96-well ELISA plate and incubated overnight at 4° C. The next day, after washing twice with PBS, the wells were blocked with 3% BSA, 100 μL per well, and incubated at 37° C. for 1.5 hours, followed by washing 4 times with PBST. Gradient diluted TIGIT monoclonal antibodies and CD155-mFC (6 μg/mL) (Human CD155 (Fc Tag): Sino Biological, 10109-H02H) were mixed in equal volumes and added to the blocked and washed microplate and incubated at 37° C. for 2 hours, followed by 4 washes with PBST. Subsequently, a diluted secondary antibody HRP-conjugated anti-mFC (Jackson Immuno Research, 115-035-164) was added and incubated at 37° C. for 1.5 hours. After washing 4 times with PBST, TMB coloring solution was added, and the plate was incubated at 37° C. for 10⁻¹⁵ minutes before reading on a microplate reader (wavelengths 450 nm and 655 nm).

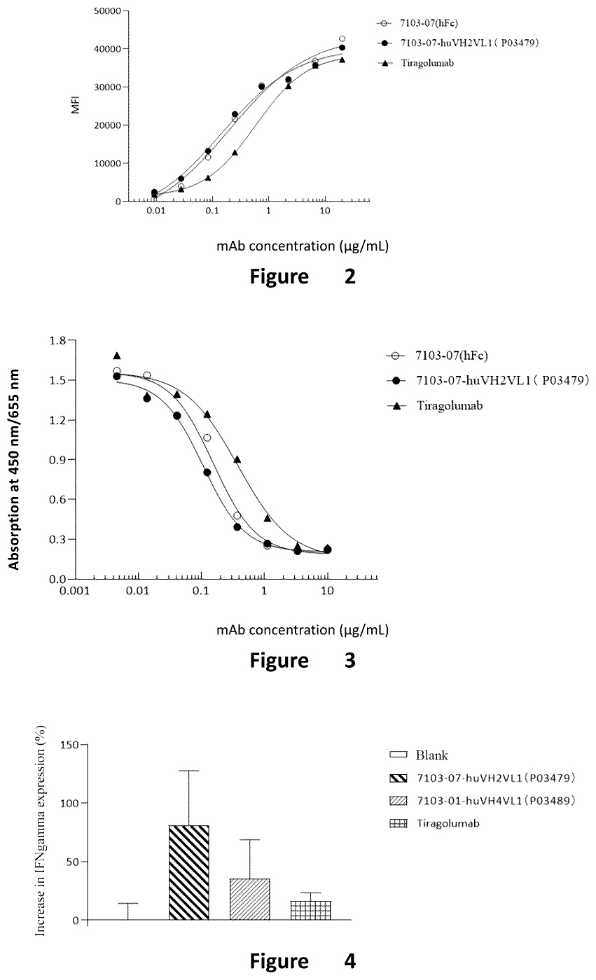

FIG. 3 shows the results of the ELISA study. As shown, all TIGIT monoclonal antibodies could specifically block the binding between TIGIT and CD155. The humanized mAb P03479 showed inhibitory activity with an IC50 of 0.107 μg/mL (0.714 nM), which was comparable to or better than the IC₅₀ value of the human-mouse chimeric monoclonal antibody (7103-07 (hFc)) at 0.154 μg/mL (1.033 nM), and significantly better than the IC₅₀ value of Tiragolumab at 0.378 μg/mL (2.534 nM).

### 1.5 In Vitro Enhancement of INFγ Production in Human PBMCs

Following T lymphocyte activation, lymphocytes secreted the cytokine IFNγ. To test the function of TIGIT monoclonal antibodies as positive regulators of T cell activation, we conducted the following experiment to show the enhancing effect of humanized TIGIT monoclonal antibodies on IFNγ production in PBMC cells due to TIGIT signal blockade. The method is summarized as follows: Thawed human peripheral blood mononuclear cells (PBMCs) were revived and the cell density was adjusted to 5×10⁶ cells/mL. RPMI 1640+10% FBS medium was used to dilute the tested TIGIT antibodies to a concentration of 20 μg/mL, and PHA to a concentration of 6 μg/mL. Then, 50 μL of PHA dilution and 50 μL of antibody dilution were added to each well of a 96-well plate, followed by the addition of 100 μL of cell suspension to each well. After gentle mixing, the plates were incubated at 37° C. in a 5% CO₂ incubator for 4 days. After 4 days, 100 μL of supernatant was collected to measure the IFNγ content.

FIG. 4 shows the results. As demonstrated, the humanized TIGIT monoclonal antibodies P03479 and P03489 enhanced the release of IFNγ from PHA-activated PBMCs. The IFNγ release levels with humanized TIGIT antibodies P03479 and P03489 added were 81% and 36% higher than the blank (without humanized TIGIT monoclonal antibodies), respectively, and both were more effective than the 16% increase observed with Tiragolumab.

### 1.6 In Vivo Functional Study of Inhibiting Tumor Growth in Mice

Since the antibodies of this invention do not bind to mouse TIGIT, we used human TIGIT gene knock-in mice (C57BL/6) to study the in vivo antitumor effects of the antibodies. Human TIGIT gene knock-in mice were housed in an SPF-level environment. In this example, the mouse tumor model was colon cancer MC38. The method is summarized as follows: MC38 colon cancer tumor cells were obtained from Shanghai Southern Model Biotechnology Co., Ltd. Twenty-four human TIGIT gene knock-in C57/B6 mice, aged 7-9 weeks, were divided into three groups (4 males and 4 females/group) and inoculated subcutaneously in the axilla with 1×10⁶ MC38 cells per mouse. On the day of inoculation (DO), the mice in each group were administered PBS (control group), recombinant human IgG1 at 10 mg/kg (control group), and humanized TIGIT antibody P03479 at 10 mg/kg via intraperitoneal injection. The administration was performed twice a week for a total of four times. Tumor volume (length×width²/2) and mouse weight were measured at each administration. The experiment was terminated on D17. The mice were euthanized by cervical dislocation, and the tumors were weighed.

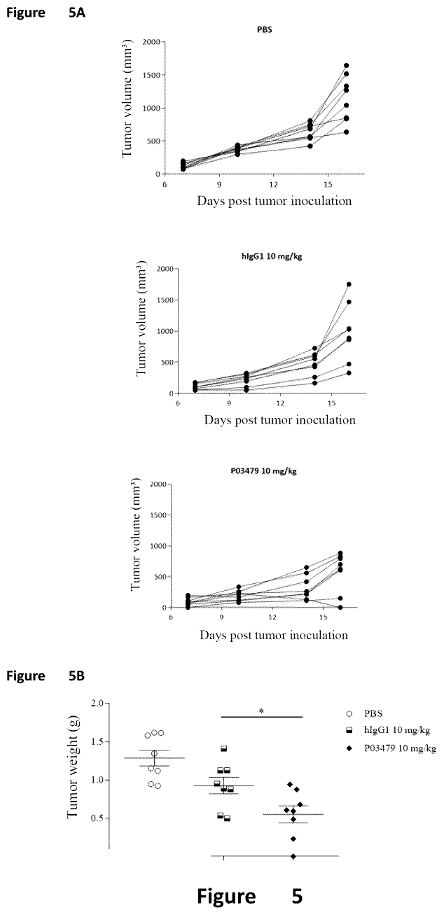

The study results are shown in FIG. 5. FIG. 5A depicts the relationship between tumor volume and time for each mouse in all groups, while FIG. 5B shows the tumor weight of each mouse in all groups at the end of the experiment (D17). The results indicate that the humanized TIGIT antibody P03479 effectively inhibited the growth of mouse tumors (FIGS. 5A and 5B). At the end of the experiment (D17), the average tumor weight in the P03479 group was significantly lower than in the hIgG1 group (p<0.05).

### 2. Antibody Modification

To reduce the antibody-mediated ADCC and CDC activities, amino acid mutations D265A and N297G (DANG) were introduced into the Fc region of the antibody (EU numbering), reducing the antibody's binding to Fc gamma receptors (FcγRs) and C1q (Lund et al. (1996) J. Immunol. 157, 4963-4969; Tao and Morrison, (1989) J. Immunol. 143, 2595-2601). The gene of the modified antibody was obtained through chemical synthesis (GeneWiz, China; same below).

### 3. Wild-Type IL2 and Mutant IL2

The full-length gene of wild-type human IL2 (WT) (including the signal peptide sequence, SEQ ID NO: 96) was chemically synthesized. The region of IL2 that binds to the IL2Rα subunit differs from the region that binds to the IL2Rβ subunit. Introducing amino acid mutations into these two regions of IL2 can alter the affinity of IL2 binding to the IL2Rα and IL2Rβ subunits. IL2 mutants (L80F, R81D, L85V, 186V, 192F) have increased affinity for binding to the IL2Rβ subunit (Levin et al. (2012) Nature 484, 529-533), and the IL2 mutant (F42A) has decreased affinity for binding to the IL2Rα subunit. The genes for the IL2 mutants (L80F, R81D, L85V, 186V, 192F) were chemically synthesized, and then site-directed mutagenesis was used to introduce the amino acid mutation (F42A) into this gene sequence, resulting in the construction of an IL2mut gene (SEQ ID NO: 98) that encodes an IL2 mutant with decreased affinity for the IL2Rα subunit and increased affinity for the IL2Rβ subunit.

The characteristics of IL2mut include the following mutations:
a) F42A—Eliminates IL2/IL2Rα interaction
b) L80F, R81D, L85V, 186V, I92F—Enhances IL2/IL2Rβ interaction.

### 4. Construction of the Fusion Protein Expression Vector

IL2 or its mutants were fused to either the N-terminus or C-terminus of one of the heavy chains of the antibody through a linker peptide GSGGGGS. To ensure that only one of the two heavy chains of the antibody was fused with IL2, we employed the “knob-into-hole” (KiH) technology (Merchant et al (1998) Nat Biotech 16, 677-681), or the electrostatic steering interaction-based technology (ART-Ig) (PCT/JP2006/306803), to maximally produce heterodimeric antibodies.

Briefly speaking, the KiH technology involves introducing mutations in the regions wherein the Fc parts of the two heavy chains closely interact, with one heavy chain incorporating mutations that form a knob (S354C and T366W in EU numbering) and the other heavy chain incorporating mutations that form a hole (Y349C, T366S, L368A, Y407V in EU numbering), thereby producing a more structurally stable heterodimeric antibody.

The ART-Ig technology, in brief, involves introducing mutations of positive and negative charges in the regions wherein the Fc parts of the two heavy chains closely interact, with one heavy chain incorporating positive charge amino acid mutations (ART-Ig-P) (E356K and H435R in EU numbering) and the other heavy chain incorporating negative charge amino acid mutations (ART-Ig-N) (K439E in EU numbering), thereby producing a more structurally stable heterodimeric antibody. IL2 can be fused to either of the two heavy chains. In the present disclosure, IL2 is fused to the antibody heavy chain HC (hole) or HC (ART-Ig-P).

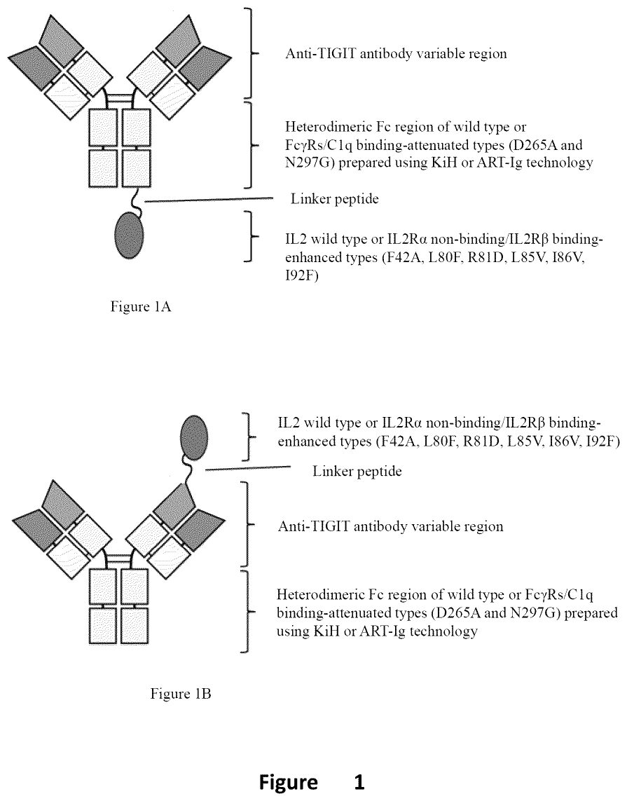

The genes for the anti-TIGIT antibody heavy chain and IL2 were amplified separately by PCR, and then the IL2 gene was linked to the N-terminus or C-terminus of the antibody heavy chain using the overlapping extension PCR method, constructing IL2-HC or TIGIT HC-IL2 genes (see the schematic diagrams in FIGS. 1A and 1B, respectively). HC1 represents the heavy chain of the humanized TIGIT antibody P03479, and HC2 represents the heavy chain of the humanized TIGIT antibody P03489.

TABLE 5
Nucleic acid molecules of IL2 fused to the N-terminus of the TIGIT
antibody heavy chain and corresponding amino acid sequences

| Nucleic acid | IL2 fused to the N-terminus | Amino acid |
|---|---|---|
| SEQ ID NO: | of antibody heavy chain | SEQ ID NO: |
| 1 | IL2wt-HC1 (hole) | 39 |
| 3 | IL2wt-HC1 (ART-Ig-P) | 42 |
| 5 | IL2mut-HC1 (hole) | 44 |
| 6 | IL2mut-HC1 (ART-Ig-P) | 45 |
| 7 | IL2mut-HC2 (hole) | 46 |
| 9 | IL2mut-HC2 (ART-Ig-P) | 49 |
| 11 | IL2wt-HC1(DANG-hole) | 51 |
| 13 | IL2wt-HC1 (DANG-ART-Ig-P) | 53 |
| 15 | IL2mut-HC1 (DANG-hole) | 55 |
| 16 | IL2mut-HC1 (DANG-ART-Ig-P) | 56 |
| 17 | IL2mut-HC2 (DANG-hole) | 57 |
| 19 | IL2mut-HC2 (DANG-ART-Ig-P) | 59 |

TABLE 6
Nucleic acid molecules of the fusion protein antibody heavy
and light chains and corresponding amino acid sequences

| Nucleic acid | | Amino acid |
|---|---|---|
| SEQ ID NO: | Antibody heavy and light chains | SEQ ID NO: |
| 2 | HC1 (knob) | 40 |
| 4 | HC1 (ART-Ig-N) | 43 |
| 8 | HC2 (knob) | 47 |
| 10 | HC2 (ART-Ig-N) | 50 |
| 12 | HC1 (DANG-knob) | 52 |
| 14 | HC1 (DANG-ART-Ig-N) | 54 |
| 18 | HC2 (DANG-knob) | 58 |
| 20 | HC2 (DANG-ART-Ig-N) | 60 |
| | LC1 | 41 |
| | LC2 | 48 |

TABLE 7
Nucleic acid molecules of IL2 fused to the C-terminus of the TIGIT
antibody heavy chain and corresponding amino acid sequences

| Nucleic acid | IL2 fused to the C-terminus | Amino acid |
|---|---|---|
| SEQ ID NO: | of antibody heavy chain | SEQ ID NO: |
| 25 | HC1(hole)-IL2wt | 66 |
| 26 | HC1(ART-Ig-P)-IL2wt | 67 |
| 27 | HC1(hole)-IL2mut | 68 |
| 28 | HC1(ART-Ig-P)-IL2mut | 69 |
| 29 | HC2(hole)-IL2mut | 70 |
| 30 | HC2(ART-Ig-P)-IL2mut | 71 |
| 31 | HC1(DANG-hole)-IL2wt | 72 |
| 32 | HC1(DANG-ART-Ig-P)-IL2wt | 73 |
| 33 | HC1(DANG-hole)-IL2mut | 74 |
| 34 | HC1(DANG-ART-Ig-P)-IL2mut | 75 |
| 35 | HC2(DANG-hole)-IL2mut | 76 |
| 36 | HC2(DANG-ART-Ig-P)-IL2mut | 77 |

Additionally, IL2mut-IgG1 HC or IgG1 HC-IL2mut constructs were constructed as control samples for the TIGIT antibody-IL2 fusion proteins of the present disclosure. IgG1 can be any human or humanized anti-human protein antibody. In the present disclosure, the IgG1 used was a humanized anti-TNFR2 antibody:

TABLE 8
Control molecule nucleic acid molecules
and corresponding amino acid sequences

| Nucleic acid | | Amino acid |
|---|---|---|
| SEQ ID NO: | Recombinant moleucle | SEQ ID NO: |
| 21 | IL2mut-IgG1 HC(ART-Ig-P) | 61 |
| 22 | IgG1 HC(ART-Ig-N) | 62 |
| 23 | IL2mut-IgG1 HC(DANG-ART-Ig-P) | 63 |
| 24 | IgG1 HC(DANG-ART-Ig-N) | 64 |
| 37 | IgG1 HC(ART-Ig-P)-IL2mut | 78 |
| 38 | IgG1 HC(DANG-ART-Ig-P)-IL2mut | 79 |

TABLE 9
Nucleic acid molecule sequences and amino acid sequences
for IL-2 wild type (wt) and mutant (mut)

| | Amino acid | | Nucleic acid |
|---|---|---|---|
| SEQ ID NO: | molecule | SEQ ID NOs | molecule |
| 96 | IL-2wt | 97 | IL-2wt |
| 98 | IL-2mut | 99 | IL-2mut |

The aforementioned constructed genes were cloned into the mammalian cell expression vector pKD2.4, which comprised a CMV promoter and a transcription termination BGH-polyA signal sequence. The cloning sites were NotI and XbaI.

### Example 2. Preparation of Anti-TIGIT Antibody-IL2 Fusion Protein

The expression plasmids constructed as described above were co-transfected into mammalian human 293F cells in an appropriate combination for the transient expression of various anti-TIGIT antibody-IL2 fusion proteins (using Sinobio transient expression kits), or to create stably expressing CHO cell lines. The transfected cells or cells stably expressing the protein were cultured in serum-free medium, and then the antibody-IL2 fusion proteins were purified from the culture supernatant using a protein A affinity chromatography column. The purified antibody fusion proteins were characterized by SDS-PAGE and SEC-HPLC analysis.

TABLE 10
Anti-TIGIT antibody-IL2 fusion proteins prepared using the KiH technology

| SEQ ID | | SEQ ID | |
|---|---|---|---|
| NO: | Fusion protein | NO: | Fusion protein |
| 39 | IL2wt-HC1/LC1(KiH) | 66 | HC1-IL2wt/LC1(KiH) |
| 40 | | 40 | |
| 41 | | 41 | |
| 51 | IL2wt-HC1(DANG)/LC1(KiH) | 72 | HC1(DANG)-IL2wt/LC1(KiH) |
| 52 | | 52 | |
| 41 | | 41 | |
| 44 | IL2mut-HC1/LC1(KiH) | 68 | HC1-IL2mut/LC1(KiH) |
| 40 | | 40 | |
| 41 | | 41 | |
| 55 | IL2mut-HC1(DANG)/LC1(KiH) | 74 | HC1(DANG)-IL2mut/LC1(KiH) |
| 52 | | 52 | |
| 41 | | 41 | |
| 46 | IL2mut-HC2/LC2(KiH) | 70 | HC2-IL2mut/LC2(KiH) |
| 47 | | 47 | |
| 48 | | 48 | |
| 57 | IL2mut-HC2(DANG)/LC2(KiH) | 76 | HC2(DANG)-IL2mut/LC2(KiH) |
| 58 | | 47 | |
| 48 | | 48 | |

TABLE 11
Anti-TIGIT Antibody-IL2 Fusion Proteins
Prepared Using the ART-Ig Technology

| SEQ ID | | SEQ ID | |
|---|---|---|---|
| NO: | Fusion protein | NO: | Fusion protein |
| 42 | IL2wt HC1/LC1(ART-Ig) | 67 | HC1-IL2wt/LC1(ART-Ig) |
| 43 | | 43 | |
| 41 | | 41 | |
| 53 | IL2wt | 73 | HC1(DANG)-IL2wt/LC1(ART-Ig) |
| 54 | HC1(DANG)/LC1(ART-Ig) | 54 | |
| 41 | | 41 | |
| 45 | IL2mut HC1/LC1(ART-Ig) | 69 | HC1-IL2mut/LC1(ART-Ig) |
| 43 | | 43 | |
| 41 | | 41 | |
| 56 | IL2mut | 75 | HC1(DANG)-IL2mut/LC1(ART-Ig) |
| 54 | HC1(DANG)/LC1(ART-Ig) | 54 | |
| 41 | | 41 | |
| 49 | IL2mut HC2/LC2(ART-Ig) | 71 | HC2-IL2mut/LC2(ART-Ig) |
| 50 | | 50 | |
| 48 | | 48 | |
| 59 | IL2mut | 77 | HC2(DANG)-IL2mut/LC2(ART-Ig) |
| 60 | HC2(DANG)/LC2(ART-Ig) | 60 | |
| 48 | | 48 | |

TABLE 12
Control Antibody-IL2mut Fusion Proteins Prepared
Using the ART-Ig Technology are as follows:

| SEQ ID | | SEQ ID | |
|---|---|---|---|
| NO: | Fusion protein | NO: | Fusion protein |
| 61 | IL2mut-IgG1 HC/LC(ART-Ig) | 78 | IgG1 HC-IL2mut/LC(ART-Ig) |
| 62 | | 62 | |
| 65 | | 65 | |
| 63 | IL2mut-IgG1 | 79 | IgG1 |
| 64 | HC(DANG)/LC(ART-Ig) | 64 | HC(DANG)-IL2mut/LC(ART-Ig) |
| 65 | | 65 | |

In the tables above, HC1 and HC2 respectively represent the heavy chains of the humanized antibodies P03479 and P03489.

The SDS-PAGE characterization of these proteins is shown in FIG. 6, wherein lanes 1, 4, and 6 respectively represent the electrophoresis under non-reducing conditions of HC1-IL2 wt/LC1 (ART-Ig), HC1-IL2mut/LC1 (ART-Ig), and IgG1 HC-IL2mut/LC (ART-Ig). Lanes 2, 5, and 7 respectively represent the electrophoresis under reducing conditions. Lane 3 shows the electrophoresis of human IgG1 under reducing conditions. The electrophoresis of the three fusion proteins under reducing conditions all displayed three bands: the band with the smallest molecular weight represents the normal light chain, approximately 24 kDa; the band with the largest molecular weight represents the heavy chain fused with IL2 at the C-terminus, approximately 65 kDa; and the middle band represents the other heavy chain of the heterodimeric antibody, approximately 49 kDa.

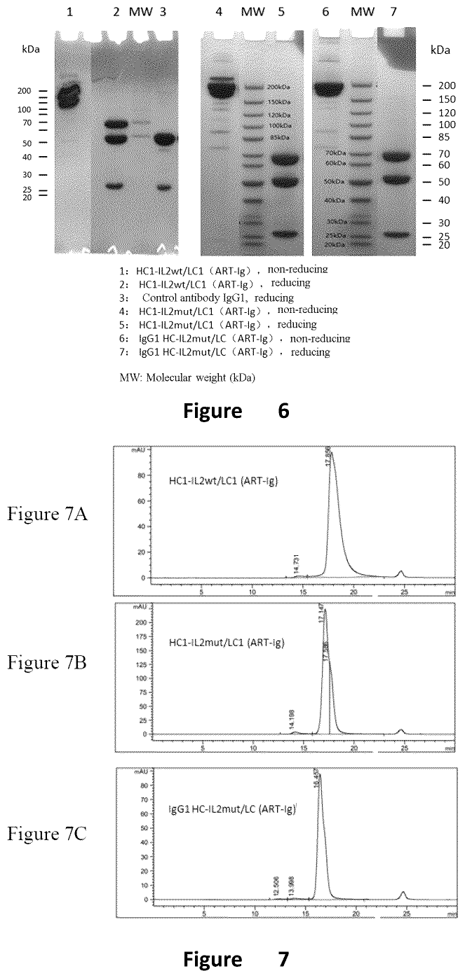

The SEC-HPLC analysis is shown in FIG. 3. The chromatograms for HC1-IL2 wt/LC1 (ART-Ig), HC1-IL2mut/LC1 (ART-Ig), and IgG1 HC-IL2mut/LC (ART-Ig) are depicted in FIGS. 7A, 7B, and 7C, respectively. The results indicate that the obtained antibody-IL2 fusion proteins appear as dimeric antibody forms in the SEC-HPLC analysis, with no polymers detected.

These results demonstrate that the desired fusion proteins were successfully prepared.

### Example 3. Affinity Testing of Fusion Proteins for Recombinant IL2Rα and IL2Rβ Subunits In Vitro

The binding affinity of the purified anti-TIGIT antibody-IL2 fusion proteins to the recombinant IL2Rα and IL2 subunits (Sinobio) was tested using Bio-Layer Interferometry (BLI).

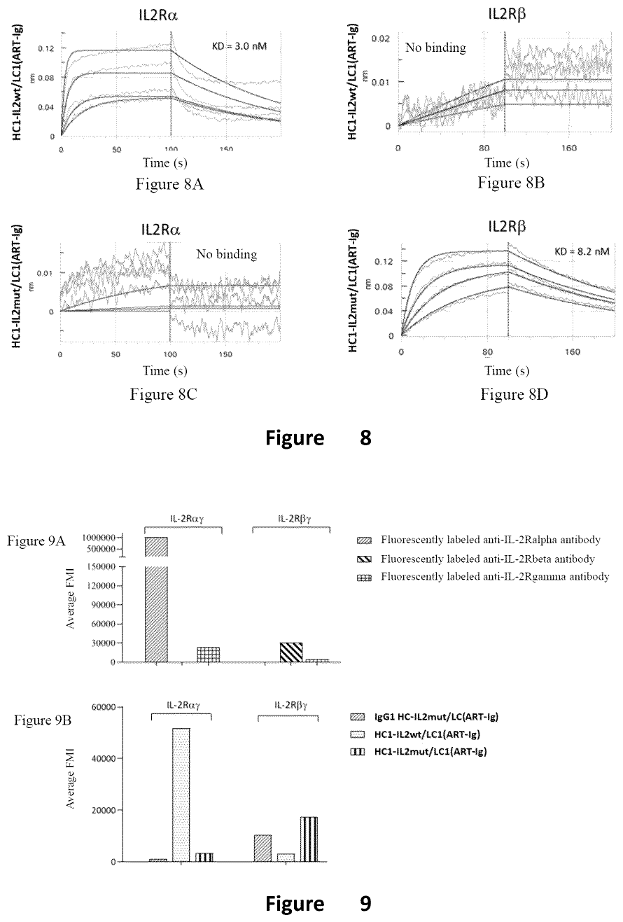

FIG. 8 shows the BLI results for the binding affinity of HC1-IL2 wt/LC1 (ART-Ig) (SEQ ID NOs: 67, 43, 41) and HC1-IL2mut/LC1 (ART-Ig) (SEQ ID NOs: 69, 43, 41) to the IL2Rα and IL2Rβ subunits, respectively.

The results indicate that HC1-IL2 wt/LC1 (ART-Ig) has a strong affinity for the IL2Rα subunit, with a binding constant KD of approximately 3.0 nM (FIG. 8A), while its affinity for the IL2Rβ subunit is weak (almost undetectable) (FIG. 8B). In contrast with HC1-IL2 wt/LC1 (ART-Ig), HC1-IL2mut/LC1 (ART-Ig) has a weaker affinity for the IL2Rα subunit (FIG. 8C) and a stronger affinity for the IL2Rβ subunit, with a binding constant KD of approximately 8.2 nM (FIG. 8D).

The BLI detection results are consistent with our expectations, showing that HC1-IL2 wt/LC1 (ART-Ig) binds strongly to the IL2Rα subunit but weakly to the IL2Rβ subunit. Conversely, HC1-IL2mut/LC1 (ART-Ig) exhibits the opposite pattern, with strong binding affinity to IL2Rβ and the binding to IL2Rα subunit being abolished.

### Example 4. Testing the Affinity of Fusion Proteins for Cell Membrane-Bound IL2Rα and IL2Rβ Subunits In Vitro

The binding affinity of the anti-TIGIT antibody-IL2 fusion proteins to the IL2Rα and IL2Rβ subunits present on the cell membrane was tested using flow cytometry.

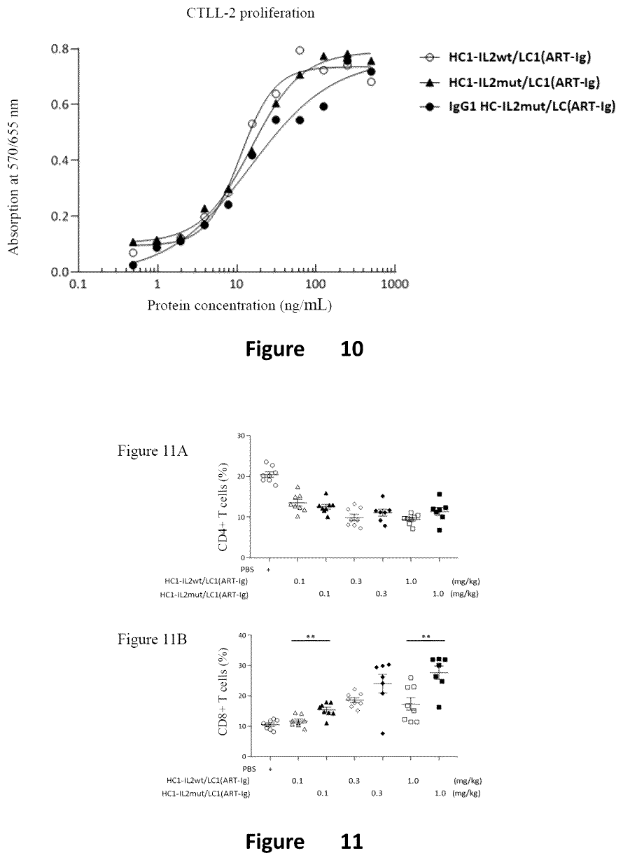

First, human IL2Rα, IL2Rβ, and IL2Rγ expression plasmids (Sinobio) were co-transfected into 293F cells to transiently express the following IL2R complexes: IL2Rαγ (dimer) and IL2Rβγ (dimer). The expression of the respective IL2R subunits on the cell membrane was detected using antibodies against IL2Rα, IL2Rβ, and IL2Ry. Flow cytometry results showed that IL2Rαγ and IL2Rβγ were expressed on the membrane of 293F cells (FIG. 9A).

Then, the binding activity of HC1-IL2 wt/LC1 (ART-Ig), HC1-IL2mut/LC1 (ART-Ig), and IgG1 HC-IL2mut/LC (ART-Ig) to the 293F cells expressing the two types of IL2R complexes (ay and By) was detected using flow cytometry. FIG. 9B shows the flow cytometry results. The results indicate that HC1-IL2 wt/LC1 (ART-Ig) binds strongly to IL2Rαγ but weakly to the IL2Rβγ subunit. In contrast with HC1-IL2 wt/LC1 (ART-Ig), both HC1-IL2mut/LC1 (ART-Ig) and IgG1 HC-IL2mut/LC (ART-Ig) exhibit weak affinity to IL2Rαγ and stronger binding to IL2Rβγ.

The flow cytometry detection of the binding activity of HC1-IL2 wt/LC1 (ART-Ig), HC1-IL2mut/LC1 (ART-Ig), and IgG1 HC-IL2mut/LC (ART-Ig) to the cell membrane-bound IL2Rαγ and the dimeric IL2Rβγ aligns with our expectations and is consistent with the BLI detection results. Specifically, HC1-IL2 wt/LC1 (ART-Ig) shows strong affinity to the IL2Rαγ subunit but weak affinity to the IL2Rβγ subunit. The antibody-IL2mut fusion proteins demonstrate strong binding affinity to IL2Rβγ, with significantly reduced or even abolished binding to IL2Rαγ.

### Example 5. Study on the Biological Function of IL2 Fusion Proteins in Stimulating the Proliferation of CTLL-2 Cells In Vitro

IL2 is one of the important growth factors for lymphocyte growth and can stimulate the proliferation of various lymphocytes, including the mouse T lymphocyte cell line CTLL-2. Human IL2 can bind to mouse IL2R with an affinity comparable to that for human IL2R. We used CTLL-2 cells to test the biological activity of HC1-IL2 wt/LC1 (ART-Ig) and HC1-IL2mut/LC1 (ART-Ig) in stimulating lymphocyte growth.

CTLL-2 cells (obtained from the National Institutes for Food and Drug Control, China) were cultured in RPMI1640 medium (Gibco) comprising 10% FBS (fetal bovine serum) (Gibco) and recombinant human (rh) IL2 (Beijing Double-Crane Pharmaceutical Co., Ltd.). The day before the experiment, 1×10⁴ cells were added to each well of a 96-well cell culture plate, comprising 0.2 mL of medium (10% FBS, without rhIL2), followed by the addition of serially diluted anti-TIGIT antibody-IL2 fusion proteins to be tested, HC1-IL2 wt/LC1 (ART-Ig), and HC1-IL2mut/LC1 (ART-Ig), along with the control antibody IgG1-HC-IL2mut/LC (ART-Ig) fusion protein. Two days later, cell growth was assessed using the CCK-8 cell growth assay kit.

The results (FIG. 10) showed that the HC1-IL2mut/LC1 (ART-Ig) fusion protein comprising IL2mut and the IgG1-HC-IL2mut/LC (ART-Ig) fusion protein stimulated CTLL-2 cell growth with EC50 values of 15.3 ng/mL and 14.8 ng/mL, respectively, which were not significantly different from the biological activity EC50 (11.1 ng/ml) of the IgG1-HC-IL2 wt/LC (ART-Ig) fusion protein comprising IL2 wt. This indicates that IL2mut possesses good activity in stimulating lymphocyte growth in vitro. The study of CTLL-2 cells expressing IL-2Rαβγ showed that the binding activity of IL2 wt and IL2mut to IL-2Rαβγ did not differ.

### Example 6. Study of Fusion Proteins Binding to Cell Membrane-Bound TIGIT

The binding activity of the anti-TIGIT antibody-IL2 fusion proteins to TIGIT on the cell membrane was tested using flow cytometry. A Jurkat cell line stably expressing human TIGIT was first established. The full-length human TIGIT gene (Sinobio) was amplified by PCR and cloned into the mammalian cell expression vector pcDNA3.1 (Invitrogen), using Not I and Xba I as cloning enzymes. The TIGIT expression plasmid was transfected into Jurkat cells to construct a stable Jurkat cell line expressing TIGIT (TIGIT-Jurkat).

The flow cytometry study method for the binding of TIGIT antibody-IL2 fusion proteins to cell membrane-bound TIGIT is summarized as follows: FACS buffer (1×PBS+0.5% BSA solution) was prepared. The required number of TIGIT-Jurkat cells, about 1×10⁵ to 5×10⁵ cells per sample, was taken and incubated with HC1-IL2 wt/LC1 (ART-Ig) and HC1-IL2mut/LC1 (ART-Ig) diluted to a certain ratio in FACS buffer, approximately 100 μL, at 4° C. for 30 minutes. After washing the cells once with FACS buffer, they were resuspended in FITC-conjugated anti-human IgG (Abcam: 6854) diluted in FACS buffer and incubated at 4° C. for 30 minutes. Finally, after washing the cells with FACS buffer, they were resuspended in 200 μL of FACS buffer for analysis. The parent TIGIT antibody P03479 was used as a control.

Flow cytometry showed that both HC1-IL2 wt/LC1 (ART-Ig) and HC1-IL2mut/LC1 (ART-Ig) could bind to TIGIT-Jurkat, with EC50 values of 0.28 μg/mL and 0.33 μg/mL, respectively, comparable to the EC50 of 0.26 μg/mL for the parent TIGIT antibody P03479.

### Example 7. Study on the Effect of Fusion Proteins on the Growth of CD4+ and CD8+ Lymphocytes In Vivo

We used human TIGIT knock-in mice to study the in vivo stimulation effect of the anti-TIGIT-IL2 fusion proteins of this invention on the growth of T lymphocytes, specifically CD4+ and CD8+ cells.

Fifty-six C57BL/6 mice (Biocytogen, 8-10 weeks old, half male and half female) were divided into 7 groups (8 per group) and injected intravenously with different doses of the fusion proteins HC1-IL2 wt/LC1 (ART-Ig) and HC1-IL2mut/LC1 (ART-Ig) or PBS (control group). The doses of the fusion proteins were 0.1 mg/kg, 0.3 mg/kg, and 1 mg/kg, with an injection volume of 0.2 mL. Four days later, blood was collected from the orbital sinus, and fluorescently labeled anti-mouse CD4 or CD8 antibodies were added. After incubating for 1 hour, the cells were washed three times with PBS and then analyzed by flow cytometry to measure the fluorescence intensity and calculate the content of CD4+ and CD8+ cells in the blood.

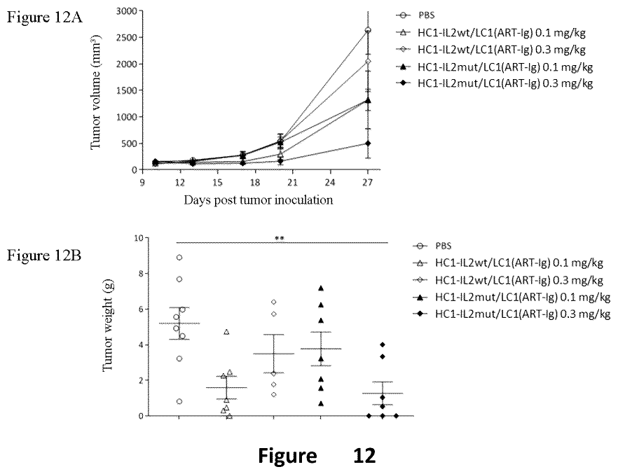

FIG. 11A shows that the content of CD4+ cells in the blood of mice treated with the fusion proteins was reduced compared to the control group mice, but there was no significant difference between the effects of HC1-IL2 wt/LC1 (ART-Ig) and HC1-IL2mut/LC1 (ART-Ig) on CD4+ cells.

FIG. 11B shows that the content of CD8+ cells in the blood of mice treated with the fusion proteins increased compared to the mice in the control group. The 0.1 mg/kg dose of HC1-IL2 wt/LC1 (ART-Ig) had no effect on the content of CD8+ cells, and only higher doses increased the content of CD8+ cells. However, HC1-IL2mut/LC1 (ART-Ig) increased the content of CD8+ cells even at low doses, with a very significant increase at higher doses. The effect of 0.1 mg/kg and 1 mg/kg doses of HC1-IL2mut/LC1 (ART-Ig) on the content of CD8+ cells was significantly different from that of the same doses of HC1-IL2mut/LC1 (ART-Ig), with a highly significant statistical difference (p<0.01).

### Example 8. Study on Inhibition of Tumor Growth In Vivo

We used human TIGIT knock-in mice to study the function of the anti-TIGIT antibody-IL2 fusion proteins of this invention in inhibiting the growth of syngeneic tumors.

Forty C57BL/6 mice (Biocytogen, 8-10 weeks old, half male and half female) were divided into 5 groups (8 per group) and were intravenously injected with different doses of the fusion proteins HC1-IL2 wt/LC1 (ART-Ig) and HC1-IL2mut/LC1 (ART-Ig), or PBS (control group). The doses of the fusion proteins were 0.1 mg/kg and 0.3 mg/kg, with an injection volume of 0.2 mL. Four days later (D4), the mice were subcutaneously inoculated with mouse colon cancer cells MC38, 1×10⁶ cells per mouse. Then, on D14, D17, and D24, the mice received a second, third, and fourth dose of the fusion proteins or PBS. The experiment ended on D31, and the tumors were weighed.

The results are shown in FIG. 12. FIG. 12A shows the relationship between the average tumor weight in each group and time, and FIG. 12B shown the tumor weight in each group at the end of the experiment (D31). The result showed that HC1-IL2mut/LC1 (ART-Ig) significantly inhibited or delayed the growth of MC38 tumors. Compared to the control group, the tumor growth was inhibited by 27% and 70% at doses of 0.1 mg/kg and 0.3 mg/kg, respectively (p<0.01) (FIG. 12B). In the group receiving 0.3 mg/kg, tumors were completely eradicated in 3 mice, and the inhibitory effect of HC1-IL2mut/LC1 (ART-Ig) on tumor growth was dose-dependent. The 0.1 mg/kg dose of HC1-IL2 wt/LC1 (ART-Ig) could inhibit the growth of MC38 tumors, but the higher dose (0.3 mg/kg) had almost no inhibitory effect, and there was no dose-response relationship in the effect of HC1-IL2 wt/LC1 (ART-Ig) on tumor growth.

All references mentioned in this application are incorporated herein by reference as if each individual publication was specifically and individually indicated to be incorporated by reference. Furthermore, it should be understood that after reading the teachings of this application, those skilled in the art may make various changes or modifications to the application, and these equivalent forms likewise fall within the scope defined by the claims attached hereto.

What is claimed is:

1. A fusion protein, comprising:
(a) a first polypeptide, comprising an anti-TIGIT antibody or antigen-binding fragment thereof;
(b) a second polypeptide, comprising interleukin-2 (IL-2) or a variant thereof having a lymphocyte growth promoting activity,
wherein the second polypeptide is fused to the first polypeptide.

2. The fusion protein according to claim 1, wherein the first polypeptide comprises an anti-TIGIT antibody or antigen-binding fragment thereof selected from the group consisting of:
(a) the anti-TIGIT antibody or antigen-binding fragment thereof that competitively binds to TIGIT with CD155 and has a binding affinity to human TIGIT with a EC50 of 0.01-20 nM; and/or
(b) the anti-TIGIT antibody or antigen-binding fragment thereof comprising heavy chain complementarity-determining regions (VH CDRs) 1-3 and light chain complementarity-determining regions (VL CDRs) 1-3, selected from the group consisting of:
VH CDR1 selected from the group consisting of: SEQ ID NO: 81 or 89;
VH CDR2 selected from the group consisting of: SEQ ID NO: 82 or 90;
VH CDR3 selected from the group consisting of: SEQ ID NO: 83 or 91;
VL CDR1 selected from the group consisting of: SEQ ID NO: 85 or 93;
VL CDR2 selected from the group consisting of: SEQ ID NO: 86 or 94; and
VL CDR3 selected from the group consisting of: SEQ ID NO: 87 or 95; and/or
(c) the anti-TIGIT antibody or antigen-binding fragment thereof comprising amino acid sequences of VH CDRs 1-3 as follows: SEQ ID NOs: 81, 82, and 83; or SEQ ID NOs: 89, 90, and 91; and amino acid sequences of VL CDRs 1-3 as follows: SEQ ID NOs: 85, 86, and 87; or SEQ ID NOs: 93, 94, and 95; and/or
(d) the anti-TIGIT antibody or antigen-binding fragment thereof comprising VH CDRs 1-3 and VL CDRs 1-3, selected from the group consisting of: SEQ ID NOs: 81, 82, and 83 and SEQ ID NOs: 85, 86, and 87; or SEQ ID NOs: 89, 90, and 91 and SEQ ID NOs: 93, 94, and 95; and/or
(e) the anti-TIGIT antibody or antigen-binding fragment thereof comprising a heavy chain variable region VH, selected from the group consisting of: SEQ ID NO: 80 or 88; and/or a light chain variable region VL, selected from the group consisting of: SEQ ID NO: 84 or 92; and/or
(f) the anti-TIGIT antibody or antigen-binding fragment thereof comprising a combination of VH and VL, selected from the group consisting of: VH of SEQ ID NO: 80 and VL of SEQ ID NO: 84; or VH of SEQ ID NO: 88 and VL of SEQ ID NO: 92, or
a sequence having at least 95% sequence identity thereto.

3. The fusion protein according to claim 1, wherein the first polypeptide comprises an IgG1 gamma constant region (e.g., IgG1 gamma constant region with accession number UniProtKB-P01857), the constant region has characteristics selected from the group consisting of:
(a) the constant region does not comprise mutations;
(b) the constant region comprises one or more mutations that reduce antibody-dependent cell-mediated cytotoxicity (ADCC) and complement-dependent cytotoxicity (CDC) activities, such as amino acid mutations D265A and N297G;
(c) the constant region comprises a pair of heavy chain constant regions (CHs), one of which comprises a mutation that introduces a knob structure (e.g., mutations S354C and/or T366W), and the other CH comprises a mutation that introduces a hole structure (e.g., Y349C, T366S, L368A, and/or Y407V), wherein the knob structure matches the hole structure to form a stable dimer;
(d) the constant region comprises a pair of CHs, one CH comprises an amino acid mutation that introduces a positive charge (e.g., mutations E356K and H435R), and the other CH comprises an amino acid mutation that introduces a negative charge (e.g., K439E), wherein a stable dimer is formed through electrostatic interaction,
wherein the positions of the above mutations are according to the EU numbering.

4. The fusion protein according to claim 1, wherein the second polypeptide has one or more characteristics selected from the group consisting of:
(a) the second polypeptide further comprises a signal peptide;
(b) the IL2 is wild-type IL2 or its functional fragment, such as derived from humans, primates, rodents;
(c) compared to wild-type IL2, the IL2 variant has increased binding affinity to IL-2Rβ subunit and/or reduced binding affinity to IL-2Rα subunit;
(d) compared to wild-type IL2, the IL2 variant has unchanged or enhanced lymphocyte growth promoting activity.

5. The fusion protein according to claim 1, wherein the IL2 variant comprises one or more mutations selected from the group consisting of: L80F, R81D, L85V, 186V, 192F, F42A, for example, comprises a combination of mutations L80F, R81D, L85V, 186V, and 192F and/or mutation F42A, wherein the positions of the above mutations are according to the EU numbering.

6. The fusion protein according to claim 1, wherein the first and second polypeptides are linked by a linker, for example, the linker is a glycine linker, like Gₙ, or a glycine/serine linker, such as amino acid sequences (GS)ₙ, (GGS)ₙ, (GGGS)ₙ, (GGGGS)ₙ, or (GGGGGS)ₙ, wherein n is an integer of 1, 2, 3, 4, 5, 6, 7, 8, 9, or 10; and/or
the second polypeptide is connected to the N-terminus and/or C-terminus of the antibody heavy chain in the first polypeptide.

7. The fusion protein according to claim 1, wherein the heavy chain of the first polypeptide is fused to the second polypeptide to form an amino acid sequence selected from the group consisting of: SEQ ID NOs: 39, 51, 44, 55, 46, 57, 66, 72, 68, 74, 70, 76, 42, 53, 45, 56, 49, 59, 67, 73, 69, 75, 71, and 77, or a sequence having at least 80% sequence identity to said sequence; and/or
if present, the heavy chain in the fusion protein not fused to the second polypeptide has an amino acid sequence selected from the group consisting of: SEQ ID NOs: 40, 52, 47, 58, 43, 54, 50, and 60, or a sequence having at least 80% sequence identity to said sequence; and/or
if present, the light chain in the fusion protein has an amino acid sequence selected from the group consisting of: SEQ ID NOs: 41 and 48, or a sequence having at least 80% sequence identity to said sequence.

8. The fusion protein according to claim 1, wherein the fusion protein includes a sequence combination selected from the group consisting of:
(a) SEQ ID NOs: 39, 40, and 41; (b) SEQ ID NOs: 51, 52, and 41;
(c) SEQ ID NOs: 44, 40, and 41; (d) SEQ ID NOs: 55, 52, and 41;
(e) SEQ ID NOs: 46, 47, and 48; (f) SEQ ID NOs: 57, 58, and 48;
(g) SEQ ID NOs: 66, 40, and 41; (h) SEQ ID NOs: 72, 52, and 41;
(i) SEQ ID NOs: 68, 40, and 41; (j) SEQ ID NOs: 74, 52, and 41;
(k) SEQ ID NOs: 70, 47, and 48; (1) SEQ ID NOs: 76, 47, and 48;
(a′) SEQ ID NOs: 42, 43, and 41; (b′) SEQ ID NOs: 53, 54, and 41;
(c′) SEQ ID NOs: 45, 43, and 41; (d′) SEQ ID NOs: 56, 54, and 41;
(e′) SEQ ID NOs: 49, 50, and 48; (f) SEQ ID NOs: 59, 60, and 48;
(g′) SEQ ID NOs: 67, 43, and 41; (h′) SEQ ID NOs: 73, 54, and 41;
(i′) SEQ ID NOs: 69, 43, and 41; (j′) SEQ ID NOs: 75, 54, and 41;
(k′) SEQ ID NOs: 71, 50, and 48; (l′) SEQ ID NOs: 77, 60, and 48.

9. An isolated nucleic acid molecule or a construct or vector comprising the nucleic acid molecule, wherein the nucleic acid molecule encodes the fusion protein of any one of claims 1-8.

10. The nucleic acid molecule, construct, or vector according to claim 9, comprising a nucleotide sequence selected from SEQ ID NOs: 1-20 or SEQ ID NOs: 25-36, or sequences having more than 90% homology thereto and having the same biological activity.

11. A cell comprising the fusion protein of any one of claims 1-8, or the nucleic acid molecule, construct, or vector of claim 9 or 10.

12. A composition comprising the fusion protein of any one of claims 1-8, the nucleic acid molecule, construct, or vector of claim 9 or 10, or the cell of claim 11; and a carrier.

13. Use of the fusion protein of any one of claims 1-8, the nucleic acid molecule, construct, or vector of claim 9 or 10, the cell of claim 11, or the composition of claim 12 in the preparation of a medicament for immunotherapy.

14. The use according to claim 13, wherein the medicament is for positively regulating immune cell activity and/or enhancing immune response; and/or the medicament is for treating cancer, immunodeficiency disease, inflammatory disease, or infectious disease.

15. The use according to claim 14, wherein:
the cancer is selected from: bladder cancer, breast cancer, uterine cancer, endometrial cancer, ovarian cancer, colorectal cancer, colon cancer, head and neck cancer, lung cancer, stomach cancer, germ cell tumor, bone cancer, squamous cell carcinoma, skin cancer, melanoma, central nervous system tumor, sarcoma, virus-associated cancer, small cell lung cancer, non-small cell lung cancer, pancreatic cancer, glioblastoma, glioma, cervical cancer, liver cancer, salivary gland cancer, kidney cancer, basal cell carcinoma, prostate cancer, vulvar cancer, thyroid cancer, testicular cancer, esophageal cancer, nasopharyngeal carcinoma, malignant pleural mesothelioma, Hodgkin's or non-Hodgkin's lymphoma, myeloma, leukemia, myelodysplastic syndrome, and any combination thereof;
the inflammatory disease, autoimmune disease, or pathogen infection disease is selected from: type 1 diabetes, multiple sclerosis, rheumatoid arthritis, celiac disease, systemic lupus erythematosus, lupus nephritis, cutaneous lupus, idiopathic arthritis, Crohn's disease, ulcerative colitis or systemic sclerosis, graft versus host disease, psoriasis, alopecia areata, atopic dermatitis, HCV-induced vasculitis, Sjögren's syndrome, pemphigus, ankylosing spondylitis, Behcet's disease, Wegener's granulomatosis, autoimmune hepatitis, sclerosing cholangitis, Gu-Syndrome and macrophage activation syndrome, autoimmune thyroiditis, autoimmune uveitis, aplastic anemia, HIV, viral hepatitis, HTLV-1 virus infection, lymphocytic choriomeningitis virus infection, parasitic infections (Echinococcosis, Schistosomiasis, Malaria).

16. A method for producing the fusion protein of any one of claims 1-8, the method comprising: culturing the cell of claim 11 under conditions suitable for expressing the fusion protein; and recovering the fusion protein.

17. An anti-TIGIT antibody or antigen-binding fragment thereof, comprising:
(a) heavy chain complementarity-determining regions (VH CDRs) 1-3 and light chain complementarity-determining regions (VL CDRs) 1-3 selected from the group consisting of:
VH CDR1 selected from the group consisting of: SEQ ID NO: 81 or 89;
VH CDR2 selected from the group consisting of: SEQ ID NO: 82 or 90;
VH CDR3 selected from the group consisting of: SEQ ID NO: 83 or 91;
VL CDR1 selected from the group consisting of: SEQ ID NO: 85 or 93;
VL CDR2 selected from the group consisting of: SEQ ID NO: 86 or 94; and
VL CDR3 selected from the group consisting of: SEQ ID NO: 87 or 95; and/or
(b) amino acid sequences of VH CDRs 1-3 as follows: SEQ ID NOs: 81, 82, and 83; or SEQ ID NOs: 89, 90, and 91; and amino acid sequences of VL CDRs 1-3 as follows: SEQ ID NOs: 85, 86, and 87; or SEQ ID NOs: 93, 94, and 95; and/or
(c) VH CDRs 1-3 and VL CDRs 1-3 selected from the group consisting of: SEQ ID NOs: 81, 82, and 83 and SEQ ID NOs: 85, 86, and 87; or SEQ ID NOs: 89, 90, and 91 and SEQ ID NOs: 93, 94, and 95; and/or
(d) a heavy chain variable region VH selected from the group consisting of: SEQ ID NO: 80 or 88; and/or a light chain variable region VL selected from the group consisting of: SEQ ID NO: 84 or 92; and/or
(e) a combination of VH and VL selected from the group consisting of: VH of SEQ ID NO: 80 and VL of SEQ ID NO: 84; or VH of SEQ ID NO: 88 and VL of SEQ ID NO: 92.

18. The anti-TIGIT antibody or antigen-binding fragment thereof according to claim 17, further comprising an IgG1 gamma constant region (e.g., IgG1 gamma constant region with accession number UniProtKB-P01857), the constant region has characteristics selected from the group consisting of:
(a) the constant region does not comprise mutations;
(b) the constant region comprises one or more mutations that reduce antibody-dependent cell-mediated cytotoxicity (ADCC) and complement-dependent cytotoxicity (CDC) activities, such as amino acid mutations D265A and N297G;
(c) the constant region comprises a pair of heavy chain constant regions (CHs), one of which comprises a mutation that introduces a knob structure (e.g., mutations S354C and/or T366W), and the other CH comprises a mutation that introduces a hole structure (e.g., Y349C, T366S, L368A, and/or Y407V), wherein the knob structure matches the hole structure to form a stable dimer,
(d) the constant region comprises a pair of CHs, one CH comprises an amino acid mutation that introduces a positive charge (e.g., mutations E356K and H435R), and the other CH comprises an amino acid mutation that introduces a negative charge (e.g., K439E), wherein a stable dimer is formed through electrostatic interaction,
wherein the positions of the mutations are according to the EU numbering.


SEQUENCE LISTING (99 sequences)

SEQ ID NO: 1 - DNA, 1788 bp, ARTIFICIAL
  IL2wt- HC1(hole)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccagga

SEQ ID NO: 2 - DNA, 1344 bp, ARTIFICIAL
  HC1(knob)
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatgtcgggaggaaatgaccaagaaccaggtcagtctgtggtgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagga

SEQ ID NO: 3 - DNA, 1788 bp, ARTIFICIAL
  IL2wt- HC1(ART-Ig-P)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 4 - DNA, 1344 bp, ARTIFICIAL
  HC1(ART-Ig-N)
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcaggaatcactgtcactgtccccaggg

SEQ ID NO: 5 - DNA, 1788 bp, ARTIFICIAL
  IL2mut- HC1(hole)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccagga

SEQ ID NO: 6 - DNA, 1788 bp, ARTIFICIAL
  IL2mut- HC1(ART-Ig-P)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 7 - DNA, 1788 bp, ARTIFICIAL
  IL2mut-HC2(hole)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccagga

SEQ ID NO: 8 - DNA, 1344 bp, ARTIFICIAL
  HC2(knob)
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatgtcgggaggaaatgaccaagaaccaggtcagtctgtggtgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagga

SEQ ID NO: 9 - DNA, 1788 bp, ARTIFICIAL
  IL2mut-HC2(ART-Ig-P)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 10 - DNA, 1344 bp, ARTIFICIAL
  HC2(ART-Ig-N)
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcaggaatcactgtcactgtccccaggg

SEQ ID NO: 11 - DNA, 1788 bp, ARTIFICIAL
  IL2wt-HC1(DANG-hole)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccagga

SEQ ID NO: 12 - DNA, 1344 bp, ARTIFICIAL
  HC1(DANG-knob)
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatgtcgggaggaaatgaccaagaaccaggtcagtctgtggtgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagga

SEQ ID NO: 13 - DNA, 1788 bp, ARTIFICIAL
  IL2wt-HC1(DANG-ART-Ig-P)
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 14 - DNA, 1344 bp, ARTIFICIAL
  HC1(DANG-ART-Ig-N)
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcaggaatcactgtcactgtccccaggg

SEQ ID NO: 15 - DNA, 1788 bp, ARTIFICIAL
  IL2mut-HC1(DANG-hole)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccagga

SEQ ID NO: 16 - DNA, 1788 bp, ARTIFICIAL
  IL2mut-HC1(DANG-ART-Ig-P)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatccgaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 17 - DNA, 1788 bp, ARTIFICIAL
  IL2mut-HC2(DANG-hole)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccagga

SEQ ID NO: 18 - DNA, 1344 bp, ARTIFICIAL
  HC2(DANG-knob)
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatgtcgggaggaaatgaccaagaaccaggtcagtctgtggtgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcagaaatcactgtcactgtccccagga

SEQ ID NO: 19 - DNA, 1788 bp, ARTIFICIAL
  IL2mut-HC2(DANG-ART-Ig-P)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 20 - DNA, 1344 bp, ARTIFICIAL; identical to SEQ ID NO: 10
  HC2(DANG-ART-Ig-N)

SEQ ID NO: 21 - DNA, 1788 bp, ARTIFICIAL
  IL-2mut-IgG1 HC(ART-Ig-P)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctccgtgaagctgtcctgcaaggcttctggctacaccttcatcacctacgacatcaactgggtccgacaggctcctggacagggactcgaatggatcggctggatctaccccagagatggcaacacccagttcaacgagaacttcaagggcaaagccaccatgaccgtggacgagtctacctccaccgcctacatggaagtgcggtccctgagatctgacgacaccgccgtgtactactgcgccagaggcgatagagactacggctacttcgatgtgtggggccagggcaccacagtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 22 - DNA, 1788 bp, ARTIFICIAL
  IgG1 HC(ART-Ig-N)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctccgtgaagctgtcctgcaaggcttctggctacaccttcatcacctacgacatcaactgggtccgacaggctcctggacagggactcgaatggatcggctggatctaccccagagatggcaacacccagttcaacgagaacttcaagggcaaagccaccatgaccgtggacgagtctacctccaccgcctacatggaagtgcggtccctgagatctgacgacaccgccgtgtactactgcgccagaggcgatagagactacggctacttcgatgtgtggggccagggcaccacagtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcaggaatcactgtcactgtccccaggg

SEQ ID NO: 23 - DNA, 1788 bp, ARTIFICIAL
  IL2mut-IgG1 HC(DANG-ART-Ig-P)
gcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgaccggcggcggcggctctggcggaggtggcagcggcggtggcggatcccaggttcagctggttcagtctggcgccgaagtgaagaaacctggcgcctccgtgaagctgtcctgcaaggcttctggctacaccttcatcacctacgacatcaactgggtccgacaggctcctggacagggactcgaatggatcggctggatctaccccagagatggcaacacccagttcaacgagaacttcaagggcaaagccaccatgaccgtggacgagtctacctccaccgcctacatggaagtgcggtccctgagatctgacgacaccgccgtgtactactgcgccagaggcgatagagactacggctacttcgatgtgtggggccagggcaccacagtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccaggg

SEQ ID NO: 24 - DNA, 1344 bp, ARTIFICIAL
  IgG1 HC(DANG-ART-Ig-N)
caggttcagctggttcagtctggcgccgaagtgaagaaacctggcgcctccgtgaagctgtcctgcaaggcttctggctacaccttcatcacctacgacatcaactgggtccgacaggctcctggacagggactcgaatggatcggctggatctaccccagagatggcaacacccagttcaacgagaacttcaagggcaaagccaccatgaccgtggacgagtctacctccaccgcctacatggaagtgcggtccctgagatctgacgacaccgccgtgtactactgcgccagaggcgatagagactacggctacttcgatgtgtggggccagggcaccacagtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcgggaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcactacactcaggaatcactgtcactgtccccaggg

SEQ ID NO: 25 - DNA, 1788 bp, ARTIFICIAL
  HC1(hole)IL2wt
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccaggaggcggcggcggctctggcggaggtggcagcggcggtggcggatccgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 26 - DNA, 1788 bp, ARTIFICIAL
  HC1(ART-Ig-P)IL2wt
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 27 - DNA, 1788 bp, ARTIFICIAL
  HC1(hole) IL2mut
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccaggaggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 28 - DNA, 1788 bp, ARTIFICIAL
  HC1(ART-Ig-P)IL2mut
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 29 - DNA, 1788 bp, ARTIFICIAL
  HC2(hole)IL2mut
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccaggaggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 30 - DNA, 1788 bp, ARTIFICIAL
  HC2(ART-Ig-P)IL2mut
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 31 - DNA, 1788 bp, ARTIFICIAL
  HC1(DANG-hole) IL2wt
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccaggaggcggcggcggctctggcggaggtggcagcggcggtggcggatccgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 32 - DNA, 1788 bp, ARTIFICIAL
  HC1(DANG-ART-Ig-P)IL2wt
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 33 - DNA, 1788 bp, ARTIFICIAL
  HC1(DANG-hole) IL2mut
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccaggaggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 34 - DNA, 1788 bp, ARTIFICIAL
  HC1(DANG-ART-Ig-P)IL2mut
gaggtgcaggttgtggaatctggcggaggactggttaagcctggcggctctctgagactgtcttgtgccgcttctggcttcaccttctccacctacgccatgtcctgggtccgacaggctcctggcaaaggactggaatgggtcgccgagatttcttccggcggctcccacaccttttacgccgataccgtgaagggcagattcaccatctctcgggacaacgccaagaacaccctgtacctgcagatgaactccctgagagccgaggacaccgccgtgtactactgtgccagaaagaccctggactactacgccctggattactggggccagggcaccacagtgacagtgtcatctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 35 - DNA, 1788 bp, ARTIFICIAL
  HC2(DANG-hole) IL2mut
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtgcactctgcctccatctcgggaggaaatgaccaagaaccaggtcagtctgtcatgtgctgtgaaaggcttctatccctccgacatcgcagtggagtgggaaagcaatggacagcctgagaacaattacaagaccacaccccctgtgctggactctgatggcagtttctttctggtcagtaagctgaccgtggataaatcacggtggcagcagggaaatgtctttagttgttcagtgatgcacgaagcactgcacaatcactacactcagaaatcactgtcactgtccccaggaggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 36 - DNA, 1788 bp, ARTIFICIAL
  HC2(DANG-ART-Ig-P)IL2mut
caggttcagctggttcagtctggcgccgaagtgaagaaacctggcgcctctgtgaaggtgtcctgcaaggcttccggctacaccttcatcagctacaacatctactgggtccgacaggcccctggacagggacttgaatggatgggcggagtgaacccctccaacggcaacaccaacttcaacgagaacttccagggcagagtgaccatgaccgtggacacctctatctccaccgcctacatggaactgtcccggctgagatctgacgacaccgccgtgtactactgcaccagaggcaactactacggctacgagttcgcctattggggccagggcacaaccgtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 37 - DNA, 1788 bp, ARTIFICIAL
  IgG1 HC(ART-Ig-P)IL-2mut
caggttcagctggttcagtctggcgccgaagtgaagaaacctggcgcctccgtgaagctgtcctgcaaggcttctggctacaccttcatcacctacgacatcaactgggtccgacaggctcctggacagggactcgaatggatcggctggatctaccccagagatggcaacacccagttcaacgagaacttcaagggcaaagccaccatgaccgtggacgagtctacctccaccgcctacatggaagtgcggtccctgagatctgacgacaccgccgtgtactactgcgccagaggcgatagagactacggctacttcgatgtgtggggccagggcaccacagtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggacgtgtcccacgaggaccccgaagtcaagttcaactggtacgtggatggcgtcgaagtgcataatgctaagacaaaaccaagagaggaacagtacaacagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 38 - DNA, 1788 bp, ARTIFICIAL
  IgG1 HC(DANG-ART-Ig-P)IL2mut
caggttcagctggttcagtctggcgccgaagtgaagaaacctggcgcctccgtgaagctgtcctgcaaggcttctggctacaccttcatcacctacgacatcaactgggtccgacaggctcctggacagggactcgaatggatcggctggatctaccccagagatggcaacacccagttcaacgagaacttcaagggcaaagccaccatgaccgtggacgagtctacctccaccgcctacatggaagtgcggtccctgagatctgacgacaccgccgtgtactactgcgccagaggcgatagagactacggctacttcgatgtgtggggccagggcaccacagtgacagtctcttctgcatcaacaaaggggcctagcgtgtttccactggccccctctagtaaaagcacctctggcggaacagcagccctgggttgtctggtgaaggactacttcccagagccagtcaccgtgtcctggaacagcggcgccctgacatccggagtccatacttttcctgctgtgctgcagtcatccgggctgtacagcctgagctctgtggtcactgtcccaagttcatccctgggtactcagacctatatctgcaacgtgaatcacaagccatccaataccaaagtggacaagaaagtggagcccaagagctgtgataaaacacatacttgccccccttgtcctgcaccagaactgctgggaggtccatccgtgttcctgtttccacccaagcctaaagacaccctgatgatttctcgaactccagaggtcacctgcgtggtcgtggccgtgtcccacgaggaccccgaagtcaagttcaactggtacgtggatggcgtcgaagtgcataatgctaagacaaaaccaagagaggaacagtacggcagcacttatcgcgtcgtgtctgtcctgaccgtgctgcaccaggattggctgaacggcaaggagtataagtgcaaagtgagcaataaggctctgcccgcacctatcgagaaaacaatttctaaggctaaaggacagcctagggaaccacaggtgtacactctgcctccatctcggaaggaaatgaccaagaaccaggtcagtctgacatgtctggtgaaaggcttctatccctccgacatcgcagtggagtgggaaagcaatggacagcctgagaacaattacaagaccacaccccctgtgctggactctgatggcagtttctttctgtatagtaagctgaccgtggataaatcacggtggcagcagggaaatgtctttagttgttcagtgatgcacgaagcactgcacaatcggtacactcagaaatcactgtcactgtccccagggggcggcggcggctctggcggaggtggcagcggcggtggcggatccgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc

SEQ ID NO: 39 - protein, 596 aa, ARTIFICIAL
  IL2wt- HC1(hole)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 40 - protein, 448 aa, ARTIFICIAL
  HC1(knob)
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 214 aa, ARTIFICIAL
  LC1
DVQITQSPSYLSASVGDRVTINCRASKSISKYLAWYQQKPGKAPKLLIYSGSRLQSGIPSRFSGSGYGTDFTLTISSLQPEDFATYYCQQHNEYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 596 aa, ARTIFICIAL
  IL2wt- HC1(ART-Ig-P)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 43 - protein, 448 aa, ARTIFICIAL
  HC1(ART-Ig-N)
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPG

SEQ ID NO: 44 - protein, 596 aa, ARTIFICIAL
  IL2mut- HC1(hole)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 45 - protein, 596 aa, ARTIFICIAL
  IL2mut- HC1(ART-Ig-P)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 46 - protein, 596 aa, ARTIFICIAL
  IL2mut- HC2(hole)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 47 - protein, 448 aa, ARTIFICIAL
  HC2(knob)
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 48 - protein, 214 aa, ARTIFICIAL
  LC2
DIQMTQSPSSMSASVGDRVTITCKASQHVSTAVAWYQQKPGKAPKLLIYSPSYRYTGVPSRFSGSGSGTDFTFTISSVQPEDIATYYCQQHYSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 596 aa, ARTIFICIAL
  IL2mut- HC2(ART-Ig-P)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 50 - protein, 448 aa, ARTIFICIAL
  HC2(ART-Ig-N)
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPG

SEQ ID NO: 51 - protein, 596 aa, ARTIFICIAL
  IL2wt- HC1(DANG-hole)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 52 - protein, 448 aa, ARTIFICIAL
  HC1(DANG-knob)
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 596 aa, ARTIFICIAL
  IL2wt- HC1(DANG-ART-Ig-P)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 54 - protein, 448 aa, ARTIFICIAL
  HC1(DANG-ART-Ig-N)
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPG

SEQ ID NO: 55 - protein, 596 aa, ARTIFICIAL
  IL2mut- HC1(DANG-hole)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 56 - protein, 596 aa, ARTIFICIAL
  IL2mut- HC1(DANG-ART-Ig-P)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSEVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 57 - protein, 596 aa, ARTIFICIAL
  IL2mut- HC2(DANG-hole)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 58 - protein, 448 aa, ARTIFICIAL
  HC2(DANG-knob)
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 596 aa, ARTIFICIAL
  IL2mut- HC2(DANG-ART-Ig-P)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 60 - protein, 448 aa, ARTIFICIAL
  HC2(DANG-ART-Ig-N)
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPG

SEQ ID NO: 61 - protein, 596 aa, ARTIFICIAL
  IL-2mut-IgG1 HC(ART-Ig-P)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKLSCKASGYTFITYDINWVRQAPGQGLEWIGWIYPRDGNTQFNENFKGKATMTVDESTSTAYMEVRSLRSDDTAVYYCARGDRDYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 62 - protein, 596 aa, ARTIFICIAL
  IgG1 HC(ART-Ig-N)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKLSCKASGYTFITYDINWVRQAPGQGLEWIGWIYPRDGNTQFNENFKGKATMTVDESTSTAYMEVRSLRSDDTAVYYCARGDRDYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPG

SEQ ID NO: 63 - protein, 596 aa, ARTIFICIAL
  IL2mut- IgG1 HC(DANG-ART-Ig-P)
APTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLTGGGGSGGGGSGGGGSQVQLVQSGAEVKKPGASVKLSCKASGYTFITYDINWVRQAPGQGLEWIGWIYPRDGNTQFNENFKGKATMTVDESTSTAYMEVRSLRSDDTAVYYCARGDRDYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 64 - protein, 448 aa, ARTIFICIAL
  IgG1 HC(DANG-ART-Ig-N)
QVQLVQSGAEVKKPGASVKLSCKASGYTFITYDINWVRQAPGQGLEWIGWIYPRDGNTQFNENFKGKATMTVDESTSTAYMEVRSLRSDDTAVYYCARGDRDYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQESLSLSPG

SEQ ID NO: 65 - protein, 214 aa, ARTIFICIAL
  IgG1 LC
DIQMTPSTSSLSASVGDRVTITCRASQDISNYLNWYQQKPGKAVKLLIYFKSKLHSGVPSRFSGSGSGTDFTLTISSLQPEDFATYYCQQGNTLPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 596 aa, ARTIFICIAL
  HC1(hole)IL2wt
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 67 - protein, 596 aa, ARTIFICIAL
  HC1(ART-Ig-P)IL2wt
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 68 - protein, 596 aa, ARTIFICIAL
  HC1(hole) IL2mut
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 69 - protein, 596 aa, ARTIFICIAL
  HC1(ART-Ig-P)IL2mut
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 70 - protein, 596 aa, ARTIFICIAL
  HC2(hole) IL2mut
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 71 - protein, 596 aa, ARTIFICIAL
  HC2(ART-Ig-P)IL2mut
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 72 - protein, 596 aa, ARTIFICIAL
  HC1(DANG-hole)IL2wt
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 73 - protein, 596 aa, ARTIFICIAL
  HC1(DANG-ART-Ig-P)IL2wt
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 74 - protein, 596 aa, ARTIFICIAL
  HC1(DANG-hole) IL2mut
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 75 - protein, 596 aa, ARTIFICIAL
  HC1(DANG-ART-Ig-P)IL2mut
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 76 - protein, 596 aa, ARTIFICIAL
  HC2(DANG-hole) IL2mut
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 77 - protein, 596 aa, ARTIFICIAL
  HC2(DANG-ART-Ig-P)IL2mut
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 78 - protein, 596 aa, ARTIFICIAL
  IgG1 HC(ART-Ig-P)IL-2mut
QVQLVQSGAEVKKPGASVKLSCKASGYTFITYDINWVRQAPGQGLEWIGWIYPRDGNTQFNENFKGKATMTVDESTSTAYMEVRSLRSDDTAVYYCARGDRDYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 79 - protein, 596 aa, ARTIFICIAL
  IgG1 HC(DANG-ART-Ig-P)IL2mut
QVQLVQSGAEVKKPGASVKLSCKASGYTFITYDINWVRQAPGQGLEWIGWIYPRDGNTQFNENFKGKATMTVDESTSTAYMEVRSLRSDDTAVYYCARGDRDYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNRYTQKSLSLSPGGGGGSGGGGSGGGGSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 80 - protein, 119 aa, Artificial
  P03489 7103-01 huVH4
QVQLVQSGAEVKKPGASVKVSCKASGYTFISYNIYWVRQAPGQGLEWMGGVNPSNGNTNFNENFQGRVTMTVDTSISTAYMELSRLRSDDTAVYYCTRGNYYGYEFAYWGQGTTVTVSS

SEQ ID NO: 81 - protein, 5 aa, Artificial
  7103-01 huVH4 CDR1
SYNIY

SEQ ID NO: 82 - protein, 17 aa, Artificial
  7103-01 huVH4 CDR2
GVNPSNGNTNFNENFQG

SEQ ID NO: 83 - protein, 10 aa, Artificial
  7103-01 huVH4 CDR3
GNYYGYEFAY

SEQ ID NO: 84 - protein, 107 aa, Artificial
  7103-01 huVL1
DIQMTQSPSSMSASVGDRVTITCKASQHVSTAVAWYQQKPGKAPKLLIYSPSYRYTGVPSRFSGSGSGTDFTFTISSVQPEDIATYYCQQHYSTPWTFGGGTKLEIK

SEQ ID NO: 85 - protein, 11 aa, Artificial
  7103-01 huVL1 CDR1
KASQHVSTAVA

SEQ ID NO: 86 - protein, 7 aa, Artificial
  7103-01 huVL1 CDR2
SPSYRYT

SEQ ID NO: 87 - protein, 9 aa, Artificial
  7103-01 huVL1 CDR3
QQHYSTPWT

SEQ ID NO: 88 - protein, 119 aa, Artificial
  P03479 7103-07 huVH2
EVQVVESGGGLVKPGGSLRLSCAASGFTFSTYAMSWVRQAPGKGLEWVAEISSGGSHTFYADTVKGRFTISRDNAKNTLYLQMNSLRAEDTAVYYCARKTLDYYALDYWGQGTTVTVSS

SEQ ID NO: 89 - protein, 5 aa, Artificial
  7103-07 huVH2 CDR1
TYAMS

SEQ ID NO: 90 - protein, 17 aa, Artificial
  7103-07 huVH2 CDR2
EISSGGSHTFYADTVKG

SEQ ID NO: 91 - protein, 10 aa, Artificial
  7103-07 huVH2 CDR3
KTLDYYALDY

SEQ ID NO: 92 - protein, 107 aa, Artificial
  7103-07 huVL1
DVQITQSPSYLSASVGDRVTINCRASKSISKYLAWYQQKPGKAPKLLIYSGSRLQSGIPSRFSGSGYGTDFTLTISSLQPEDFATYYCQQHNEYPWTFGGGTKLEIK

SEQ ID NO: 93 - protein, 11 aa, Artificial
  7103-07 huVL1 CDR1
RASKSISKYLA

SEQ ID NO: 94 - protein, 7 aa, Artificial
  7103-07 huVL1 CDR2
SGSRLQS

SEQ ID NO: 95 - protein, 9 aa, Artificial
  7103-07 huVL1 CDR3
QQHNEYPWT

SEQ ID NO: 96 - protein, 153 aa, Artificial
  IL-2wt
MYRMQLPSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 97 - DNA, 459 bp, Artificial
  IL-2wt
atgtacaggatgcaactcccgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtttggaggaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 98 - protein, 153 aa, Artificial
  IL-2mut
MYRMQLPSCIALSLALVTNSAPTSSSTKKTQLQLEHLLLDLQMILNGINNYKNPKLTRMLTAKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 99 - DNA, 459 bp, Artificial
  IL-2mut
atgtacaggatgcaactcccgtcttgcattgcactaagtcttgcacttgtcacaaacagtgcccctacaagcagcagcaccaagaagacacagctgcagctggagcacctgctcctggacctgcagatgatcctgaacggcatcaacaactacaagaaccctaagctgacaaggatgctcacagctaagttttacatgcctaagaaggccaccgagctgaagcacctgcagtgcctggaggaagagctgaagcctctggaagaggtgctgaacctggctcagagcaagaacttccacttcgaccctagagacgtggtgagcaacatcaacgtgttcgtgctggagctgaagggcagcgagaccaccttcatgtgcgagtacgccgacgagaccgccaccatcgtggagttcctgaacagatggatcaccttctgtcagagcatcatcagcaccctgacc